(19) United States
(12) Patent Application Publication
(10) Pub. No.: US 2025/0027032 A1
(43) Pub. Date: Jan. 23, 2025

(54) SYSTEM AND METHODS FOR COMBINED HARVEST AND CAPTURE OF A BIOLOGIC

(71) Applicant: Repligen Corporation, Waltham, MA (US)

(72) Inventors: Michael Bransby, Altadena, CA (US); Philip Yuen, Long Beach, CA (US); Westley Blacklock, Long Beach, CA (US); Marlon Tannous, Redondo Beach, CA (US); Karol Lacki, Höllviken (SE); Anthony Hunt, Boston, MA (US); James Peyser, Billerica, MA (US)

(73) Assignee: Repligen Corporation, Waltham, MA (US)

(21) Appl. No.: 18/775,747

(22) Filed: Jul. 17, 2024

Related U.S. Application Data
(60) Provisional application No. 63/514,157, filed on Jul. 18, 2023.
(60) Provisional application No. 63/652,845, filed on May 29, 2024.
(60) Provisional application No. 63/659,962, filed on Jun. 14, 2024.

(51) Int. Cl. C12M 1/00

(52) U.S. Cl. CPC C12M 47/04

(57) ABSTRACT

The invention provides tangential flow filtration systems and methods for capture and harvest of a biologic product, and related compositions.

### CROSS-REFERENCE TO RELATED APPLICATIONS

The present application claims the benefit of and priority under 35 U.S.C. § 119 to U.S. Provisional Patent Application No. 63/514,157 filed Jul. 18, 2023, U.S. Provisional Patent Application No. 63/652,845 filed May 29, 2024, and U.S. Provisional Patent Application No. 63/659,962 filed Jun. 14, 2024, and incorporates their disclosures herein by reference in their entirety.

### FIELD OF THE INVENTION

The invention relates to bioprocessing systems and methods for the manufacture of biologic products produced by cultured cells.

### BACKGROUND

In the biotechnology and pharmaceutical industries, a number of different process operations are generally used in the purification of cell-produced biologic products from bioreactor systems. These may include centrifugation, multiple filtration operations, and one or more chromatography operations. It is generally the case that each purification operation lowers product yield. Methods are needed to streamline the purification process to increase yield and decrease time and attendant costs, while maintaining high product yield and purity.

### BRIEF SUMMARY

The present invention provides methods for performing a combined capture and harvest operation initiated by a direct addition of a capture resin to a cell culture fluid or lysate, without the need for prior clarification steps such as prior centrifugation and/or filtration step. The ensuing steps utilize a combination of diafiltration and concentration operations through a tangential flow filtration device to produce a biologic product at high yield and of high purity. In aspects, direct addition of resin to cell culture fluid or lysate containing a biologic product includes contacting the cell culture fluid or lysate with the resin in a secondary vessel or in a recirculating retentate fluid circuit of a tangential flow filtration module, which may also be referred to herein for conciseness as a “recirculation loop”.

In one aspect, provided is a method for isolating a biologic product from a process fluid characterized by high cell density and/or high turbidity, comprising performing a capture operation by contacting the process fluid with a capture resin in a retentate fluid circuit of a filtration module for a period of time sufficient to allow binding of the biologic product to the resin; performing a wash operation by separating the process fluid and resin mixture into permeate and retentate fluid streams of the filtration module and recirculating the retentate fluid stream through the retentate fluid circuit for a first number of diafiltration volumes (DVs) while directing the permeate stream to a waste container outside the fluid circuit, thereby producing a clarified retentate fluid, and optionally concentrating the clarified retentate fluid in the retentate fluid circuit; performing an elution operation by contacting the clarified retentate fluid with a volume of elution buffer in the retentate fluid circuit for a period of time sufficient to allow disassociation of the biologic product from the resin; performing a harvest operation by circulating the clarified retentate fluid and resin mixture through the filtration module, thereby separating the mixture into a permeate fluid stream comprising the biologic product and a retentate stream comprising the resin; and recirculating the retentate fluid stream through the retentate fluid circuit for a second number of DVs while directing the permeate stream to a recovery container outside the fluid circuit, thereby isolating the biologic product in the recovery container.

In one aspect, provided is a method for isolating a biologic product from a process fluid characterized by high cell density and/or high turbidity, where the method includes performing a capture operation by contacting the process fluid with a capture resin in a process vessel for a period of time sufficient to allow binding of the biologic product to the resin, performing a wash operation by circulating the process fluid and resin mixture through a filtration module, thereby separating the mixture into permeate and retentate fluid streams, where the filtration module and the process vessel are interconnected in a retentate fluid circuit, and recirculating the retentate fluid stream through the retentate fluid circuit for a first number of diafiltration volumes (DVs) while directing the permeate stream to a waste container outside the fluid circuit, thereby producing a clarified retentate fluid, and optionally concentrating the clarified retentate fluid in the process vessel, performing an elution operation by contacting the clarified retentate fluid with a volume of elution buffer for a period of time sufficient to allow disassociation of the biologic product from the resin, performing a harvest operation by circulating the clarified retentate fluid and resin mixture through the filtration module, thereby separating the mixture into a permeate fluid stream includes the biologic product and a retentate fluid stream includes the resin, and recirculating the retentate fluid stream through the retentate fluid circuit for a second number of DVs while directing the permeate stream to a recovery container outside the fluid circuit, thereby isolating the biologic product in the recovery container.

In one aspect, provided is a method for isolating a biologic product from a process fluid characterized by high cell density and/or high turbidity, where the method includes performing a capture operation by contacting the process fluid with a capture resin in a process vessel for a period of time sufficient to allow binding of the biologic product to the resin while circulating the mixture of process fluid and resin through a filtration module, thereby separating the mixture into permeate and retentate fluid streams, where the filtration module and the process vessel are interconnected in a closed retentate fluid circuit and an open permeate fluid circuit such that the retentate fluid stream is prevented from exiting the filtration module, thereby retaining the resin, while the permeate fluid stream is directed through the permeate fluid circuit back to the process vessel, performing a wash operation by opening the retentate fluid circuit, circulating the process fluid and resin mixture through the filtration module and recirculating the retentate fluid stream through the retentate fluid circuit for a first number of diafiltration volumes (DVs) while closing the permeate fluid circuit and directing the permeate stream to a waste container outside the fluid circuit, thereby producing a clarified retentate fluid, and optionally concentrating the clarified retentate fluid in the process vessel, performing an elution operation by contacting the clarified retentate fluid with a volume of elution buffer for a period of time sufficient to allow disassociation of the biologic product from the resin, performing a harvest operation by circulating the clarified retentate fluid and resin mixture through the module, thereby separating the mixture into a permeate fluid stream includes the biologic product and a retentate fluid stream includes the resin, and recirculating the retentate fluid stream through the module for a second number of DVs while directing the permeate stream to a recovery container outside the fluid circuit, thereby isolating the biologic product.

In one aspect, provided is a method for isolating a biologic product from a process fluid characterized by high cell density and/or high turbidity, where the method includes performing a capture operation by (i) contacting the process fluid with a capture resin in a primary process vessel for a period of time sufficient to allow binding of the biologic product to the resin, (ii) circulating the mixture of process fluid and resin for a second period of time through a secondary process vessel in fluid communication with a filtration module, thereby separating the mixture into permeate and retentate fluid streams, where the filtration module and the secondary process vessel are interconnected in a retentate fluid circuit and the primary process vessel, secondary process vessel, and filtration module are interconnected in a permeate fluid circuit, such that the retentate fluid stream is recirculated between the secondary process vessel and filtration module in the retentate fluid circuit while the permeate fluid stream is recirculated between the primary process vessel, the secondary process vessel, and the filtration module in the permeate fluid circuit, performing a wash operation by closing the permeate fluid circuit, circulating the mixture of process fluid and resin through the filtration module and recirculating the retentate fluid stream through the retentate fluid circuit for a first number of diafiltration volumes (DVs) while directing the permeate stream to a waste container outside the fluid circuit, thereby producing a clarified retentate fluid, and optionally concentrating the clarified retentate fluid in the secondary process vessel, performing an elution operation by contacting the clarified retentate fluid with a volume of elution buffer for a period of time sufficient to allow disassociation of the biologic product from the resin, and performing a harvest operation by circulating the clarified retentate fluid and resin mixture through the filtration module, thereby separating the mixture into a permeate fluid stream includes the biologic product and a retentate fluid stream includes the resin, and recirculating the retentate fluid stream through the retentate fluid circuit for a second number of DVs while directing the permeate stream to a recovery container outside the fluid circuit, thereby isolating the biologic product.

In aspects of any of the methods described here, the filtration module comprises a large porosity tangential flow filtration (TFF) filter medium constructed of non-woven fibers having pore size in the range of 50-200 microns. In aspects, the filter medium may be in the form of a tubular/spiral flat sheet, referred to herein as a tangential flow chromatography filter or “TFCF”. In aspects, the filter medium is constructed from a nonwoven polypropylene/polyethylene polymer having a pore size of from 50-200 microns and formed into a tubular membrane, for example by spiral winding of a flat sheet membrane as illustrated in FIG. 27. Suitable membranes include nonwoven wetlaid membranes. In aspects, the TFCF medium is not formed by extrusion.

The method may also include where the capture operation is performed in a secondary vessel fluidly connected to the process vessel, which may include transferring a first volume of process fluid from the process vessel into the secondary vessel, where the secondary vessel contains the resin, or the resin is added to the secondary vessel, and performing the capture operation and wash operation to obtain a clarified retentate fluid in the secondary vessel.

The method may also include performing a second or further capture operation prior to the elution operation by transferring a second or further volume of process fluid into the secondary process vessel containing the clarified retentate fluid and performing a second or further capture operation and wash operation to obtain a second or further clarified retentate fluid in the secondary vessel, optionally repeating the capture and wash operations with a third or further volume of process fluid prior to performing the elution and harvest operations.

In all cases it is understood that a wash operation may include both diafiltration and concentration steps.

The method may also include where during one or both of the wash and harvest operations, fluid lost to the permeate stream is replaced to maintain a constant volume of fluid in the process vessel and/or the secondary vessel in either a batch or continuous process.

The method may also include where during one or both of the wash and harvest operations, fluid lost to the permeate stream is not replaced in order to concentrate the fluid in the process vessel.

The method may include where, following the harvest operation, the harvested cell product is subjected to a further filtration step comprising filtration through a tangential flow depth filter medium.

The method may also include where the process fluid is characterized by a viable cell density (VCD) or total cell density (TCD) of from 10E5 to 10E9 cells/ml for insect or mammalian cells, or by an optical density (OD) of from 1-350 at 600 or 620 nanometers (nm) for bacterial cells.

The method may also include where the process fluid is characterized by a turbidity of from 100-30,000 nephelometric turbidity units (NTUs) or from 200-1,000 NTU before contacting with the resin.

The method may also include where the process fluid is characterized by a viscosity of from about 1.5-30 centipoise (cP).

The method may also include where the wash operation is sufficient to remove 95-99% of cells and/or cellular proteins and nucleic acids from the retentate fluid stream.

The method may also include where the wash operation is sufficient to achieve a 2-5 average log reduction of cellular proteins and nucleic acids in the retentate fluid stream.

The method may also include where following the harvest operation, at least 90% of the resin is retained in the retentate fluid.

The method may also include where the filtration module includes a filtration medium comprising one or a plurality of hollow fiber elements forming a hollow fiber depth filter medium, each hollow fiber element consisting of a porous wall having a thickness of from 2-10 millimeters (mm) defining a lumen having an internal diameter (ID) of from 1-12 mm, a porosity of from about 50-90%, and a pore rating of from 10-50 microns.

The method may also include where each of the one or a plurality of hollow fiber elements forming the hollow fiber depth filter medium consists of a porous wall having a thickness of from 2-10 mm defining a lumen having an ID of from 1-12 mm, a porosity of from about 60-90%, and a pore rating of 10, 20, 30, 40, or 50 microns.

The method may also include where the filtration module includes a large porosity tangential flow chromatography or “TFC” filtration medium comprising one or a plurality of hollow fiber elements constructed of non-woven fibers having a pore size in the range of 50-200 microns. In aspects, the TFC filter medium may be in the form of a flat sheet spiral wound into a tubular form, which may also be referred to as “tubular/spiral wound”. An exemplary method for preparing a TFC filtration medium is described in FIG. 27. In aspects, the TFC filtration medium is constructed from a nonwoven polypropylene/polyethylene polymer having a pore size of from 50-200 microns. In aspects, the TFC filtration medium comprises a porous wall having a thickness of from 0.1 to 0.5 mm defining a lumen having an ID of from 1-12 mm, a porosity of from about 60-90%, and a pore rating of 10, 20, 30, 40, 50, 100, 150, or 200 microns.

The method may also include where the biologic product is an antibody, a recombinant protein or a virus particle.

The method may also include where the resin is functionalized with Fc-binding ligands or ligands that bind to virus particles. Other technical features may be readily apparent to one skilled in the art from the following figures, descriptions, and claims.

In one aspect, provided is a system for isolating a biologic product from a process fluid characterized by high cell density and/or high turbidity as defined herein, where the system includes a process vessel containing the process fluid, at least one filtration module, which may include a tangential flow filtration (TFF) or tangential flow depth filtration (TFDF) module, in fluid communication with the process vessel, where the filtration module includes one or a plurality of hollow fiber elements forming a filter medium, each hollow fiber element consisting of a porous wall having a thickness of from 0.5-10 millimeters (mm) defining a lumen having an internal diameter (ID) of from 1-12 mm, a porosity of from about 50-90% and a pore rating of from 10-50 microns, and a housing adapted to separate the process fluid into permeate and retentate fluid streams as it flows through the filter medium, the housing provided with an inlet, a permeate outlet and a retentate outlet, and at least one pump.

In one aspect, provided is a system for isolating a biologic product from a process fluid characterized by high cell density and/or high turbidity as defined herein, where the system includes a process vessel containing the process fluid, at least one filtration module in fluid communication with the process vessel, where the filtration module includes one or a plurality of hollow fiber elements forming a TFC filtration medium, each hollow fiber element consisting of a porous wall having a thickness of from 0.1-0.5 mm defining a lumen having an internal diameter (ID) of from 1-12 mm, a porosity of from about 50-90% and a pore rating of from 10-200 microns, and a housing adapted to separate the process fluid into permeate and retentate fluid streams as it flows through the filter medium, the housing provided with an inlet, a permeate outlet and a retentate outlet, and at least one pump. In aspects, the TFC filtration medium is constructed from a nonwoven polypropylene/polyethylene polymer having a pore size of from 50-200 microns, a porous wall having a thickness of from 0.1 to 0.5 mm defining a lumen having an ID of from 1-12 mm, a porosity of from about 60-90%, and a pore rating of 10, 20, 30, 40, 50, 100, 150, or 200 microns.

The system may also include where at least two modules are connected in series or in parallel with the process vessel.

The system may also include a retentate pump and a permeate pump.

The system may also include a permeate container and a product recovery container.

The system may also include a supply of fluid or buffer joined in a feed relationship to the process vessel. Other technical features may be readily apparent to one skilled in the art from the following figures, descriptions, and claims.

In one aspect, provided is a tangential flow filtration (TFF) or tangential flow depth filtration (TFDF) module which includes one or a plurality of hollow fiber elements forming a hollow fiber filter medium or hollow fiber depth filter medium, each hollow fiber element consisting of a porous wall having a thickness of from 0.5-10 mm defining a lumen having an internal diameter (ID) of from 1-12 mm, a porosity of from about 50-90%, and a pore rating of from 10-50 microns, and a housing adapted to separate the process fluid into permeate and retentate fluid streams as it flows through the filter medium, the housing provided with an inlet, a permeate outlet and a retentate outlet.

In one aspect, provided is a tangential flow filtration (TFF) module which includes one or a plurality of hollow fiber elements forming a TFC filtration medium, each hollow fiber element consisting of a porous wall having a thickness of from 0.1-0.5 mm defining a lumen having an internal diameter (ID) of from 1-12 mm, a porosity of from about 50-90%, and a pore rating of from 10-200 microns, and a housing adapted to separate the process fluid into permeate and retentate fluid streams as it flows through the filter medium, the housing provided with an inlet, a permeate outlet and a retentate outlet. In aspects, the TFC filtration medium is constructed from a nonwoven polypropylene/polyethylene polymer having a pore size of from 50-200 microns, a porous wall having a thickness of from 0.1 to 0.5 mm defining a lumen having an ID of from 1-12 mm, a porosity of from about 60-90%, and a pore rating of 10, 20, 30, 40, 50, 100, 150, or 200 microns.

### BRIEF DESCRIPTION OF THE SEVERAL VIEWS OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. In the figures, identical or nearly identical or equivalent elements are typically represented by the same reference characters, and similar elements are typically designated with similar reference numbers, with redundant description omitted. For purposes of clarity and simplicity, not every element is labeled in every figure, nor is every element of each embodiment shown where illustration is not necessary to allow those of ordinary skill in the art to understand the disclosure.

FIG. 1 is a flow chart depicting a capture/harvest operation in accordance with an aspect of the invention showing the four main processes, capture, wash, elution, and harvest.

FIG. 2 illustrates a system that may be utilized in connection with a capture/harvest operation in accordance with an aspect of the invention.

FIG. 3 illustrates a system that may be utilized in connection with a “constant feed concentration” operation in accordance with an aspect of the invention.

FIG. 4 illustrates a system that may be utilized in connection with “dead-end capture” in accordance with an aspect of the invention.

FIG. 5 is a line graph of pressure (psi) versus time (minutes).

FIG. 6 shows results of a model indicating that a 67% porous membrane would not foul at an average permeate flow of 4,000 LMH.

FIG. 7A is a line graph illustrating RNA (ng/ul) clearance from process fluid (step 1) through 9 diafiltration volumes (DVs, steps 2-10) in permeate (triangles) and retentate (squares) fluid streams through a TFDF membrane having 67% porosity.

FIG. 7B is a line graph illustrating host cell DNA (HCD, ng/ul) clearance from process fluid (step 1) through 7 diafiltration volumes (DVs, steps 2-8) in permeate (triangles) and retentate (squares) fluid streams through a TFDF membrane having 67% porosity.

FIG. 8A is a line graph illustrating turbidity (NTU) reduction starting from process fluid (step 1) through 9 diafiltration volumes (DVs, steps 2-10) in permeate (triangles) and retentate (squares) fluid streams through a TFDF membrane having 67% porosity. Turbidity is reduced to effectively zero in the permeate fluid stream. The remaining turbidity in the retentate stream is due to the resin (about 1.5% resin slurry).

FIG. 8B is a line graph of permeate absorbance (AU) during diafiltration through a TFDF membrane having 67% porosity starting from process fluid (step 1) through 9 diafiltration volumes (DVs, steps 2-10) at 260 nm (triangles) and 280 nm (squares)

FIG. 9 is a line graph of absorbance at 977 nm (AU) over time (min) for permeate (triangles) and retentate (squares) fluid streams through a TFDF membrane having 67% porosity.

FIG. 10 illustrates a system that may be utilized in connection with a “constant feed concentration” operation in accordance with an aspect of the invention.

FIG. 11 is a bar graph showing results of a harvest capture operation in accordance with an aspect of the invention.

FIG. 12 illustrates a system in accordance with an aspect of the invention.

FIG. 13 illustrates a system in accordance with an aspect of the invention.

FIG. 14 is a line graph showing percentage (%) immunoglobulin G (IgG) bound versus percentage (%) Protein A Resin (w/v) for three concentrations of IgG, 1 mg/ml IgG (top line), 3 mg/ml IgG (middle line), 10 mg/ml IgG (bottom line).

FIG. 15 is a line graph showing percentage (%) AAV capsids (AAV) bound versus percentage (%) Resin solids (w/v) for Resin 1 and Resin 2.

FIG. 16 is a line graph illustrating host cell DNA clearance in a process utilizing HEK293 cells.

FIG. 17 is a line graph illustrating lysate clearance as turbidity reduction in the permeate in a process utilizing HEK293 cells.

FIG. 18 is a bar graph showing product recovery from a 1-liter harvest-capture scale down for monoclonal antibody (mAb) harvest from Chinese Hamster Ovary (CHO) cells.

FIG. 19 illustrates the pressure profile for the 1-liter mAb harvest-capture scale down in FIG. 18.

FIG. 20 is a bar graph showing product recovery from a 1-liter harvest-capture scale down for AAV9 harvest from HEK293 cells.

FIG. 21 illustrates the pressure profile for the 1-liter AAV harvest-capture scale down in FIG. 20.

FIG. 22 is a line graph showing capture of monoclonal antibody (mAb) as mg IgG per ml resin versus time (minutes). The top curve represents measurements taken from the inline capture retentate at the indicated times; bottom curve represents measurements taken from the batch capture retentate.

FIG. 23 is a bar graph showing capture of AAV using a batch capture or inline capture process, data taken at a single 3 hour time point.

FIG. 24 is a bar graph showing percentage (%) AAV in solution for an inline single pass process for capture/harvest.

FIG. 25A shows a pressure profile for untreated lysate.

FIG. 25B shows a pressure profile for lysate treated with acidic conditions.

FIG. 25C shows a pressure profile for lysate treated with endonuclease.

FIG. 26A is a bar graph showing DNA clearance obtained with lysate treated with acidic conditions.

FIG. 26B is a bar graph showing host cell protein clearance obtained with lysate treated with acidic conditions.

FIG. 27 is a schematic illustration of a method of forming a large porosity tangential flow filtration (TFF) filter medium constructed of non-woven fibers having pore size in the range of 50-200 microns in the form of a tubular/spiral flat sheet. As illustrated, a non-woven flat strip (50-200 micron pore size) is wrapped into a spool, e.g., by spiral winding, and then thermally or ultrasonically welded to itself in a spiral pattern to form a tubular filter element. In aspects, the non-woven fiber is a polypropylene/polyethylene polymer.

### DETAILED DESCRIPTION

Recovery of cell-produced biologic products, which may include antibodies and other recombinant proteins, virus particles, viral vectors, including adeno associated virus (AAV) particles or lentivirus (LV) particles or AAV vectors, or LV vectors, as well as other nucleic acid vectors including e.g., eukaryotic or bacterial plasmid vectors, and other nucleic acid based products, including antisense oligonucleotides, mRNA, siRNA, shRNA, cDNA, etc., involves the physical separation of the biologic product from producer or host cells and/or cell debris in the cell culture fluid or lysate, abbreviated herein as “CF”. Product recovery operations are typically initiated when producer cells have reached a certain predetermined cell density, characterized as a viable cell density (VCD) or total cell density (TCD). Generally, the CF at this stage contains a high density of cells and cell debris. In operations where the cells are lysed to release the biologic product, the CF will contain high concentrations of host cell protein (HCP) and DNA (hcDNA) as well as other cell debris and/or virus particles and will typically also have a viscosity greater than 1 centipoise (cP), for example in a range of 2-30 cP, or 2-20 cP, or 2-10 cP. In either case, at the initial stage of product recovery from the CF, the CF will be characterized by high cell density or high turbidity, or both, and may also have a viscosity greater than 1 cP. Due to its high cell density and/or high turbidity, the CF is typically subjected to several clarification and concentration operations, including centrifugation, filtration, and diafiltration, to obtain a fluid of sufficient purity and concentration to be further purified by column chromatography, including affinity chromatography.

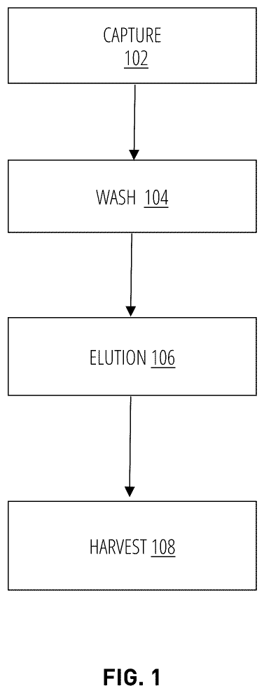

In contrast to existing methods, the present invention advantageously utilizes a single tangential flow filtration operation to perform capture and harvest of the biologic product from the CF without the need for prior clarification steps, including e.g., prior centrifugation and/or prior filtration steps. Instead, as described in detail below, an appropriate capture resin, such as an appropriately functionalized affinity resin, an ion exchange resin, a hydrophobic interaction chromatography (HIC) resin, a multimodal chromatography resin (MMC), or an immobilized metal affinity chromatography (IMAC) resin, is contacted directly with the unclarified CF under conditions permitting binding of the biologic product to the resin in a capture operation. The capture operation may also include an optional concentration step. The capture operation, which may be performed in several modes as discussed in detail infra, is followed by a wash operation (which may be preceded by an optional concentration step) which proceeds by diafiltration through a filtration module. The filtration module comprises a filter medium, which may include a tangential flow filtration (TFF) medium, a tangential flow depth filtration (TFDF) medium, or a tubular/spiral flat sheet filter medium, referred to herein as a tangential flow chromatography filter or “TFCF”. In practice, the wash operation may include a series of diafiltration and concentration steps, as described infra. Biologic product is then eluted from the capture resin and collected in a harvest operation, which in practice may also include a series of diafiltration and concentration steps, as described infra. The harvest operation also isolates the resin, which may then be regenerated. The methods and systems described here advantageously reduce process time, decrease costs and increase efficiency as well as provide high product yield due at least in part to a reduction in the number of operational steps required. In addition, the methods described here provide a biologic product of high purity. For example, as described in detail infra, where the biologic product is recombinant virus particles, the recovered virus particles have low levels of contaminating host cell protein and nucleic acids. Alternatively, where the biologic product is recombinant protein, such as an antibody or monoclonal antibody, the recovered antibody has low levels of contaminates, including viruses. FIG. 1 is a flow chart illustrating steps for performing a biologic product

capture/harvest operation in accordance with an aspect of the present invention. The term “biologic product” refers to a product produced by cells. Exemplary biologic products include recombinant proteins, antibodies, nucleic acid vectors, including viral vectors such as AAV vectors or lentiviral (LV) vectors, virus particles, including AAV and LV particles and virus-like particles (VLPs). Producer cells may be bacterial cells, yeast cells, insect cells, or mammalian cells. The biologic product may be secreted from the cells or otherwise released from the cells into the cell culture fluid (CF), for example by cell lysis. Where the cells are lysed to release the biologic product, the CF may also be referred to as the “lysate”.

An important aspect of the methods described here is that the CF has not been subjected to any clarification processes prior to contacting with an capture resin. As discussed above, the CF at this stage will be characterized by high cell density or high turbidity, or both, and may also have a viscosity greater than 1 centipoise (cP). For example, in some aspects the CF may have for a cP of from 1.5-30 cP, or 1.5-20 cP, or 1.5-10 cP, or a cP of about 1.5, about 2, about 3, about 4, abut 5, about 6, about 7, about 8, about 9, about 10, about 12, about 14, about 16, about 18, or about 20. In the following discussion, the CF is referred to as “the process fluid”. Accordingly, the process fluid at the start of a capture/harvest operation in accordance with the methods described here is characterized by a high cell density and/or high turbidity and optionally a viscosity greater than 1 cP, as defined in more detail in the following paragraphs.

In aspects where the process fluid is characterized by a high cell density, cell density may be measured as viable cell density or “VCD”, including VCD pre-lysis where the cells are lysed to release the cell product to be recovered, or total cell density “TCD” which includes both viable and non-viable cells. For example, the process fluid may have a VCD or TCD of from 1×10\hat{ }5 (10E5) to 10E9 cells per milliliter (ml). In some aspects, the process fluid may have a VCD or TCD of from about 10E5 to 10E6 cells/ml or about 10E6 to 10E7 cells/ml, or about 10E8 to 10E9 cells/ml, for mammalian and insect cells. In other aspects, for example where the cells are bacterial cells, cell density may be measured in units of optical density (OD). For example, where the cells are E. coli cells, the process fluid may have an OD of from 1-350 at 600 or 620 nm, or from 30-300 or from 30-250.

In aspects where the process fluid is characterized by a high turbidity, turbidity may be measured in nephelometric turbidity units (NTUs). In some aspects, the process fluid may have a turbidity of from about 100-30,000 NTU. In some aspects, the process fluid may have a turbidity of from about 100-10,000 NTU, or from about 100-5,000 NTU, or from about 100-2,500 NTU, or from about 100-1,000 NTU, or from about 100-500 NTU. In some aspects, the process fluid may have a turbidity of from about 200-1,000 NTU or about 300-1,000 NTU, or about 400-1,000 NTU. In some aspects, the process fluid may have a turbidity of about 300, about 400, about 500, about 600, about 700, about 800, or about 900 NTU. In this context, the turbidity of the process fluid refers to its turbidity prior to addition of the capture resin. In general, the resin may add from about 3,000-6,000 NTU or more to the fluid's turbidity, depending on the amount of resin added. The methods and systems described here allow for capture of the biologic product from highly turbid process fluids, without the need for prior clarification steps such as prior centrifugation or filtration operations.

In aspects where the process fluid is characterized by a viscosity greater than 1 cP, the process fluid may have a viscosity of from about 5-100 cP, or from 5-50 cP, or from 5-25 cP. In some aspects, the process fluid may have a viscosity of about 2, about 5, about 10, or about 15 cP. The process fluid may be characterized by a viscosity greater than 1, for example, where the cells producing the biologic product are lysed to release the biologic product into the process fluid prior to capture.

In some aspects, the process fluid is characterized by one or more of a high cell density, which may be a high viable cell density (VCD), a high total cell density (TCD) or high optical density (OD), a high turbidity, and/or a viscosity greater than 1, where high cell density, high turbidity, and viscosity are defined by the ranges discussed above.

With reference to FIG. 1, the methods described here begin with a capture operation 102. During capture, process fluid containing the biologic product as well as cells and/or cell debris, or including cell lysate where the cells are lysed to release the biologic product, and having a high cell concentration and/or high turbidity, as described above, is contacted with a capture resin for a period of time under conditions suitable for binding of the biologic product to the resin. The period of time is selected based on the particular resin in accordance with the manufacturer's recommendations. Conditions suitable for binding may include, for example, a specified temperature, in accordance with the resin manufacturer's recommendations.

In aspects where the cells are lysed in order to release biologic product, the cells may be lysed prior to initiating the capture operation using any suitable method, preferably a mechanical method such as using backpressure from a centrifugal pump.

In accordance with the methods described here, the contacting of the capture operation may be performed by direct addition of capture resin to a primary process vessel, which may be a bioreactor, or by any other suitable method, for example by addition of process fluid to a secondary process vessel containing the resin, or the resin may recirculate within a filter module flow path, also referred to as a retentate flow path. Thus, in aspects, the capture operation may be performed by addition of capture resin directly to a bioreactor or directly to a secondary process vessel in fluid communication with a bioreactor. Alternatively, the resin may reside in a retentate fluid circuit of a filtration module which is fluidly connected to a bioreactor or secondary process vessel. In accordance with any of the foregoing configurations, the retentate fluid circuit may also be fluidly connected to one or more of a wash buffer tank, an elution buffer tank, and a regeneration buffer tank. As discussed above, the term “process fluid” refers to CF, which is characterized by high cell density or high turbidity, or both, and may also have a viscosity greater than 1 cP, because the fluid has not been subjected to any prior clarification processes including prior centrifugation or filtration.

Suitable resins for use in capture of biologic product in accordance with the methods described here are in the form of beads or other particulates having a mean particle diameter that is about 1.5 to 10 times larger than the average pore size of the filter medium. Suitable capture resins include affinity resins, ion exchange resins, hydrophobic interaction chromatography (HIC) resins, multimodal chromatography resins (MMC), and immobilized metal affinity chromatography (IMAC) resins. In some aspects of the methods described here, the resin may have a mean particle size of about 20 microns, about 30 microns, or about 50 microns. In some aspects, the resin may have a mean particle size of up to 200 microns, for example from 20-50 microns or from 50-200 microns, or about 100 microns, about 150 microns, or about 200 microns. Suitable affinity resins include a chemistry or ligand chemistry capable of binding the biologic product to the resin with high affinity. For example, in some aspects the resin is functionalized with a ligand, such as Staphylococcus aureus Protein A or a derivative thereof, capable of binding the Fc region of an antibody or other Fc-containing protein. In some aspects, the resin is functionalized with a ligand capable of binding a virus particle, such as an AAV or LV particle. In some aspects, the ligand is capable of binding onc or more AAV capsid proteins. Suitable resins that may be utilized include resins formed of discrete polymeric particles functionalized with an affinity ligand where the polymeric particles may be made from a polysaccharide such as agar, agarose, dextran, starch, cellulose, pullulan, etc., and stabilized variants and derivatives thereof; or where the particles are made from a synthetic polymer such as polystyrene, polyvinylether, polyvinyl alcohol, polyacrylate, polymethacrylate, polyacrylamide, etc. Suitable affinity resins include regenerated resins and single use or disposable resins. Suitable affinity resins are commercially available. For example, AVIPure® AAV affinity resins and CaptivA® Protein A affinity resins from Repligen Corp. (Waltham MA) provide capture of AAV virus particles (also sometimes referred to in the art as AAV vectors) and Fc-containing proteins, respectively. Other suitable resins include resins functionalized with Protein A or derivatives of Protein A, such as Eshmuno® A (MilliporeSigma) and MabCaptureC® (Thermo Fisher Scientific); resins functionalized with other ligands, including metal chelates for isolation of recombinant proteins, especially histidine-tagged proteins, such as Fractogel® Metal Chelate (MilliporeSigma) or Capto® Chelating (Cytiva); and other resins functionalized with ligands for capture of virus particles, such as POROS® CaptureSelect AAV (Thermo Fisher Scientific) and Capto® AVB (Cytiva) for isolation of AAV, or CaptureSelect Lenti VSVG (Thermo Fisher Scientific) for capture of VSV-G pseudotyped LV particles.

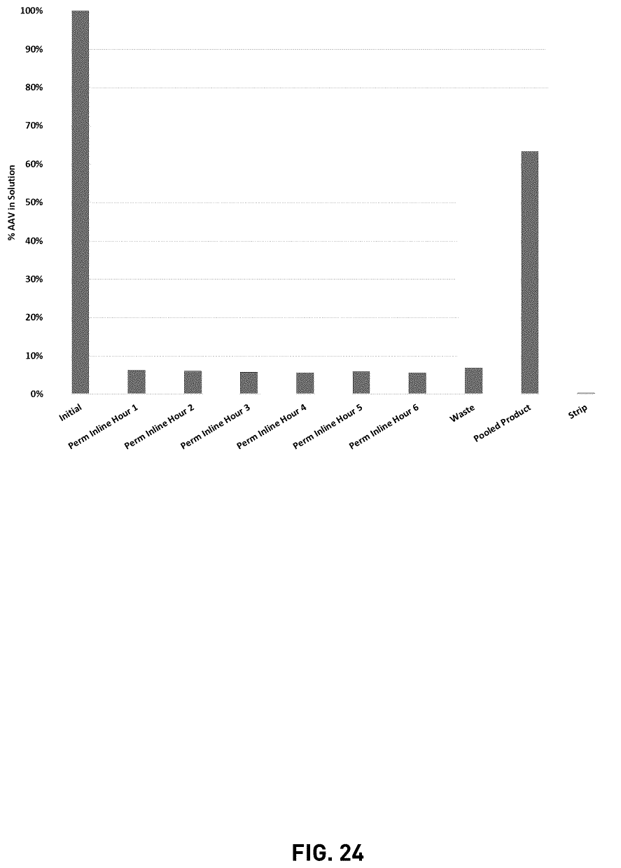

Following incubation of the capture resin with the process fluid for a period of time under conditions suitable for binding of the biologic product to the resin, a wash 104 operation is initiated to separate the resin particles from the process fluid and wash away cells, cell debris, and other contaminants, including e.g., host cell nucleic acids including DNA and RNA, host cell proteins (HCP), and virus particles where the biologic product is not the virus particles, by diafiltration through a TFF module as described herein, interconnected with the process vessel in a fluid circuit. In some aspects, the flow-through or permeate stream is returned to the bioreactor or secondary process vessel. In other aspects, the flow-through or permeate stream is sent directly to a waste container. Suitable buffers for the wash operation include, for example, include Tris, Tween, HEPES, and citric acid buffers. In some aspects, the wash buffer does not include a phosphate buffer. In some aspects, the wash buffer may include 0.15M sodium chloride (NaCl), neutral pH. In some aspects, the wash steps may be used to validate a predetermined log reduction of contaminating virus to be cleared from the final biological product. The ability to remove small viruses, i.e., virus particles having a size in the 20 nanometer range, is evidenced by the bar graph in FIG. 24. The figure shows that AAV virus passes readily through the large pore size filter. The bar labeled “strip” indicates that there is no detectable virus remaining on the resin after elution. Accordingly, a large pore size filter as described herein would also be able to pass other viruses of a similar size to AAV in order to reduce viral load in the final product. In addition, the wash buffer may include conditions for viral inactivation to inactivate enveloped virus, e.g., low pH.

A TFF module for use in the systems and methods described here comprises one or a plurality of hollow fiber elements which form a filter medium encased in a filter housing. As used herein, the term “hollow fiber” may refer to both “fibers” which are generally characterized in the industry as having lumens of less than 2 mm in diameter, and “tubes” which term may be used where the lumen has a diameter larger than 2 mm, for example in the range of 2-12 mm. Accordingly, the term “hollow fiber” is used herein to refer to fiber or tube shaped filter elements which collectively encompass lumens ranging from 1-12 mm in diameter, which is also referred to as the internal diameter or “ID” of the filter element. In some aspects, the hollow fiber elements constructed of non-woven fibers having a pore size in the range of 50-200 microns. In aspects, the filter medium may be in the form of a flat sheet spiral wound into a tubular form, which may also be referred to as “tubular/spiral wound” or in the context of the present invention, a tangential flow chromatography filter or “TFCF”. In aspects, the filtration medium is constructed from a nonwoven polypropylene/polyethylene polymer having a pore size of from 50-200 microns. Suitable membranes include nonwoven wetlaid membranes. In aspects, the TFCF medium is not formed by extrusion.

The filter housing of the TFF module includes a process fluid inlet to bring process fluid into the housing at an upstream or proximal end of the module and a retentate outlet to bring retentate fluid out of the housing from the downstream or distal end of the module. The filter housing will also include at least one permeate outlet to bring permeate fluid out of the housing. The housing may include other ports, for example a vent port and a drain port. In some aspects, the filter medium is encapsulated in the filter housing to provide an integral device that may be a single-use or disposable unit. In some aspects, the single-use or disposable unit may be sterile. In some aspects, the single-use or disposable unit may be sterilized by ethylene oxide gas sterilization or by irradiation, for example X-ray irradiation, gamma irradiation or electron beam irradiation.

Each hollow fiber element of the filter medium is comprised of a plurality of non-woven polymer fibers characterized by a pore rating of from 10-50 microns, or from 20-50 microns, or about 30 microns, about 40 microns, or about 50 microns. In aspects, the polymer fibers are sintered. In other aspects, the polymer fibers are melt-blown.

In some aspects, the hollow fiber filter medium is a “depth filter” medium and each hollow fiber element of the plurality is defined by a thick porous wall defining a lumen having an internal diameter (ID). In some aspects, the internal diameter (ID) is from about 1-12 millimeters (mm), or from about 3-6 mm, or from about 4-5 mm. In some aspects, the porous wall has a thickness of from about 2-10 mm, or from about 4-10 mm, or from about 4-6 mm, or from about 2-6 mm. In some aspects, the wall has a porosity in the range of from about 50-70% (0.50-0.70). In some aspects, the hollow fiber element(s) have a pore rating of from 10-50 microns. In some aspects, the hollow fiber element(s) have a length of from 5-150 centimeters (cm). In some aspects, the hollow fiber element(s) have a length of 5, 10, 20, 30, 40, 50, 60, 70, 80, 90, 100, 110, 120, 130, 140, or 150 cm. In some aspects, the hollow fiber element(s) have a length of about 20 cm or about 110 cm.

Porosity (P) is calculated as a weight percentage based on the density (d) of the hollow fiber element(s) measured in grams per cubic centimeter (g/cc). Where the hollow fiber element(s) consist of more than one type of polymer, a “dA” term takes into account the aggregate or blended density of the material (dA) such that porosity of the aggregate material is calculated as:

$$P = 1 - ( d / dA ) .$$

For example, where the hollow fiber element(s) are made of bi-component materials, dA is calculated as a sum of each polymer's density multiplied by its weight percentage in the material. Thus, for a material comprised of two polymers, P1 and P2, present in amounts of 70/30 weight percent, respectively, each having a density d1 and d2, respectively, the aggregate density is calculated as

$$dA = ( 0.7 \times d 1 ) + ( 0.3 \times d 2 )$$

In some aspects, the hollow fiber elements are defined by a porous wall of from about 0.075-10 mm thick, or from about 0.075-0.5 mm for a tangential flow filtration (TFF) medium and about 2-10 mm thick for a tangential flow depth filtration medium (TFDF), where the porous wall defines a lumen of from about 0.5-12 mm in diameter, where the porosity of the wall is in the range of from about 50-80% (0.50-0.80) or from about 50-70%. In some aspects, the hollow fiber element(s) have a pore rating of from 10-50 microns, or from 20-50 microns, or from 30-50 microns. In some aspects, the hollow fiber element(s) have a pore rating of about 30 microns, about 40 microns, or about 50 microns.

In some aspects, the hollow fiber tangential flow filtration (TFF) elements are defined by a thin porous wall of from about 0.075-0.3 millimeters (mm) thick. In embodiments, the porous wall defines a lumen having an ID of from about 0.5 to 6 mm in diameter or from about 0.5-2 mm in diameter, where the porosity of the wall is in the range of from about 50-80% (0.50-0.80) or from about 50-70%. In some aspects, the hollow fiber element(s) have a pore rating of from 10-50 microns, or from 20-50 microns, or from 30-50 microns. In some aspects, the hollow fiber element(s) have a pore rating of about 30 microns, about 40 microns, or about 50 microns.

In some aspects, the hollow fiber depth filtration (TFDF) elements are defined by a porous wall of from about 2-10 mm thick or about 2-6 or 4-10 mm thick, defining a lumen having an ID if from about 1-12 mm in diameter or from about 3-6 mm or about 4-5 mm in diameter, where the porosity of the wall is in the range of from about 50-80% (0.50-0.80) or from about 50-70%. In some aspects, the hollow fiber element(s) have a pore rating of from 10-50 microns, or from 20-50 microns, or from 30-50 microns. In some aspects, the hollow fiber element(s) have a pore rating of about 30 microns, about 40 microns, or about 50 microns.

The hollow fiber depth filter medium may be defined by its cross-sectional area and number of hollow fiber elements that comprise the medium, as well as by parameters of the hollow fiber element(s) that form the medium, such as the ID, wall thickness, porosity, and length of the hollow fiber element(s). In some aspects, the hollow fiber depth filter medium may be defined by its permeability in terms of its normalized water permeability (NWP) measured as LMH/psi. In some aspects, the NWP of the hollow fiber depth filter medium is from about 7,000 to about 12,000 LMH/psi.

In aspects where the filter medium is a tangential flow chromatography or “TFC” filtration medium, the hollow fiber elements are constructed of non-woven fibers having a pore size in the range of 50-200 microns. In aspects, the TFC filter medium may be in the form of a flat sheet spiral wound into a tubular form, referred to herein as a tangential flow chromatography filter or “TFCF”. In aspects, the TFCF medium is constructed from a nonwoven polypropylene/polyethylene polymer having a pore size of from 50-200 microns. In aspects, the TFCF medium includes a porous wall having a thickness of from 0.1 to 0.5 mm defining a lumen having an ID of from 1-12 mm, a porosity of from about 60-90%, and a pore rating of 10, 20, 30, 40, 50, 100, 150, or 200 microns. Suitable membranes include nonwoven wetlaid membranes. In aspects, the TFCF medium is not formed by extrusion.

In some aspects, the hollow fiber elements of the TFF or TFDF filtration media are constructed of a material that includes one or more of polysulfone, polyethersulfone (PES) or modified polyethersulfone (mPES). In embodiments, the polysulfone, PES or mPES has an anisotropic structure.

The hollow fibers for use in the filter units may be formed from a variety of materials using a variety of processes. For example, hollow fibers may be formed by assembling numerous particles, filaments, or a combination of particles and filaments into a tubular shape. The pore size and distribution of hollow fibers formed from particles and/or filaments will depend on the size and distribution of the particles and/or filaments that are assembled to form the hollow fibers. The pore size and distribution of hollow fibers formed from filaments will also depend on the density of the filaments that are assembled to form the hollow fibers. For example, mean pore sizes ranging from 0.5 microns to 50 microns may be created by varying filament density.

Suitable particles and/or filaments include both inorganic and organic particles and/or filaments. In some embodiments, the particles and/or filaments may be mono-component particles and/or mono-component filaments. In some embodiments, the particles and/or filaments may be multi-component (e.g., bi-component, tri-component, etc.) particles and/or filaments. For example, bi-component particles and/or filaments having a core formed of a first component and a coating or sheath formed of a second component, may be employed, among many other possibilities.

In various embodiments, the particles and/or filaments may be made from polymers. For example, the particles and/or filaments may be polymeric mono-component particles and/or filaments formed from a single polymer, or they may be polymeric multi-component (i.e., bi-component, tri-component, etc.) particles and/or filaments formed from two, three, or more polymers. A variety of polymers may be used to form mono-component and multi-component particles and/or filaments including polyolefins such as polyethylene and polypropylene, polyesters such as polyethylene terephthalate and polybutylene terephthalate, polyamides such as nylon 6 or nylon 66, fluoropolymers such as polyvinylidene fluoride (PVDF) and polytetrafluoroethylene (PTFE), among others. Suitable polyethylene polymers include, without limitation, high-density polyethylene (HDPE) and high- or ultra-high-molecular weight polyethylenc (UHMWPE).

Particles may be formed into tubular shapes by using, for example, tubular molds. Once formed in a tubular shape, particles may be bonded together using any suitable process. For instance, particles may be bonded together by heating the particles to a point where the particles partially melt and become bonded together at various contact points (a process known as sintering), optionally, while also compressing the particles. As another example, the particles may be bonded together by using a suitable adhesive to bond the particles to one another at various contact points, optionally, while also compressing the particles.

Filament-based fabrication techniques that can be used to form tubular shapes include, for example, simultaneous extrusion (e.g., melt-extrusion, solvent-based extrusion, etc.) from multiple extrusion dies, or electrospinning or electrospraying onto a rod-shaped substrate (which is subsequently removed), among others.

Filaments may be bonded together using any suitable process. For instance, filaments may be bonded together by heating the filaments to a point where the filaments partially melt and become bonded together at various contact points, optionally, while also compressing the filaments. As another example, filaments may be bonded together by using a suitable adhesive to bond the filaments to one another at various contact points, optionally while also compressing the filaments.

In particular embodiments, numerous fine extruded filaments may be bonded together to at various points to form a hollow fiber, for example, by forming a tubular shape from the extruded filaments and heating the filaments to bond the filaments together, among other possibilities.

In some aspects, the hollow fiber elements of the depth filter medium are formed from sintered or melt-blown polymer fibers. The terms “fibers” and “filaments” in the context of “polymer fibers” or “polymer filaments” are used interchangeably herein. Polymers that may be used include polyolefins such as polyethylene and polypropylene, polyesters such as polyethylene terephthalate and polybutylene terephthalate, polyamides such as nylon 6 or nylon 66, fluoropolymers such as polyvinylidene fluoride (PVDF) and polytetrafluoroethylene (PTFE), among others. Suitable polyethylene polymers include, high-density polyethylene (HDPE) and high- or ultra-high-molecular weight polyethylene (UHMWPE). In some aspects, the polymer is selected from polypropylene, a polyester, and mixtures thereof.

The term “sintered” in this context refers to the use of heat and optionally pressure in a bonding process. In this process, the polymer fibers are heated to a point where the filaments partially melt and become bonded together at various contact points, optionally, while also compressing the filaments. Thus, sintering bonds fibers where they touch, creating void spaces between the fibers. Numerous fine extruded filaments may be bonded together to at various points to form a hollow fiber, for example, by forming a tubular shape from the extruded filaments and heating the filaments to bond the filaments together.

The term “melt-blown” refers to the use of a gas stream at an exit of a filament extrusion die to attenuate or thin out the filaments while they are in their molten state. Melt-blown filaments are described, for example, in U.S. Pat. No. 5,607,766 to Berger. Mono- or bi-component filaments may be attenuated as they exit an extrusion die using known melt-blowing techniques to produce a collection of filaments. The collection of filaments may then be bonded together in the form of a hollow fiber.

In some aspects, hollow fibers for use in the filter medium of the filter module described here may be formed by combining bicomponent filaments having a sheath of first material which is bondable at a lower temperature than the melting point of the core material. For example, hollow fibers may be formed by combining bicomponent extrusion technology with melt-blown attenuation to produce a web of entangled biocomponent filaments, and then shaping and heating the web, for example in an oven or using a heated fluid such as steam or heated air, to bond the filaments at their points of contact. An example of a sheath-core melt-blown die is schematically illustrated in U.S. Pat. No. 5,607,766 in which a molten sheath-forming polymer and a molten core-forming polymer are fed into the die and extruded together. The molten bicomponent sheath-core filaments are extruded into a high velocity air stream, which attenuates the filaments, enabling the production of fine bicomponent filaments. U.S. Pat. No. 3,095,343 to Berger shows an apparatus for gathering and heat-treating a multi-filament web to form a continuous tubular body, such as a hollow fiber, of filaments randomly oriented primarily in a longitudinal direction, in which the body of filaments are, as a whole, longitudinally aligned and are, in the aggregate, in a parallel orientation, but which have short portions running at random in non-parallel diverging and converging directions. In this way, a web of sheath-core bicomponent filaments may be pulled into a confined area, for example by using a tapered nozzle having a central passageway forming member, where it is gathered into tubular rod shape and heated or otherwise cured to bond the filaments.

The hollow fiber depth filter medium does not have a defined pore size. However, porc size may be determined using methods known in the art, for example a “bubble point test.” The bubble point test is based on the fact that, for a given fluid and pore size, with constant wetting, the pressure required to force an air bubble through a pore is inversely proportional to the pore diameter. In practice, this means that the largest pore size of a filter can be established by wetting the filter material with a fluid and measuring the pressure at which a continuous stream of bubbles is first seen downstream of the wetted filter. The point at which a first stream of bubbles emerges from the filter material is a reflection of the largest pore(s) in the filter material, with the relationship between pressure and pore size being based on Poiseuille's law which can be simplified to P=K/d, where P is the gas pressure at the time of emergence of the stream of bubbles, K is an empirical constant dependent on the filter material, and d is porc diameter. In this regard, pore sizes determined experimentally may be measured using a device such as a POROLUX™ 1000 Porometer (Porometer NV, Belgium), or similar device.

Practically, given the large pore sizes of the filter media for use in the methods described here, a passage/retention test may be used to determine pore size, rather than a bubble point test. In accordance with the methods described here, the mean pore size of the hollow fiber element or elements forming the filter medium is selected to retain the resin particles utilized for capture of the biologic product. In some aspects, the mean pore size of the material forming the porous wall of the hollow fiber element or elements is from 1.5 to 10 times smaller, or from about 2-5 times smaller, than the mean diameter of the resin. In some aspects, the wall of the hollow fiber element(s) is characterized by a porosity of from about 50-70% (0.50-0.70), or from about 55-70%. In some aspects, the hollow fiber element(s) have a pore rating of from 10-50 microns, or from about 20 microns, about 30 microns, about 40 microns, or about 50 microns, depending on the size of the resin particles.

In some aspects, the as-formed hollow fiber element may be further coated with a suitable coating material such as PVDF either on the inside or outside of the fiber, which coating process may also act to reduce the pore size of the hollow fiber.

In some aspects, the capture resin beads have a mean diameter of 20 microns, 50 microns, or 75 microns, and the filter medium comprises hollow fiber elements in which porosity of the wall is in the range of from about 50-90% (0.50-0.90) or about 50%, about 60%, about 70%, about 80%, or about 90%. In some aspects, the hollow fiber element(s) have a pore rating of from 10-50 microns, or about 10 microns, about 20 microns, about 30 microns, about 40 microns, or about 50 microns.

Returning to FIG. 1, the wash 104 operation includes a number of diafiltration steps sufficient to remove cells, cell debris, and related impurities from the resin using a diafiltration buffer, which may for example, phosphate buffered saline (PBS) or other suitable buffer. The extent of resin washing performed during the wash operation may be measured in terms of diafiltration volumes (DVs). A single DV is the starting volume of fluid in the process vessel following capture 102. Thus, one DV has been processed when the volume of permeate is equal to equal the starting volume. Diafiltration may be performed in a batch or continuous process. In accordance with some aspects of the methods described here, diafiltration is carried out using from 5-20 DVs. The diafiltration operation is preferably performed at an average process flux of about 500-4000 LMH, or about 1000-4000 LMH

The wash 104 operation may also include concentrating the resin. In some aspects, the resin may be concentrated, for example, to about 1 to 80 percent by volume (% v/v), or from about 10-70% v/v or from about 20-60% v/v. In some aspects, the resin may be concentrated by a factor of from 10 to 100 times (10-100×).

Turning to FIG. 1, following wash 104, elution 106 is initiated by addition of an elution buffer, either to the process vessel or secondary vessel containing the resin-bound biologic product, or to the retentate fluid circuit containing the resin-bound biologic product. The elution buffer is formulated to disrupt the high affinity binding between the resin and biologic product and will be selected based on manufacturer's instructions for the particular resin utilized. Resin is incubated with elution buffer for a period of time and under conditions suitable for maximum dissociation of the biologic product from the resin. The time and conditions, which may include a specified pH and salt concentration, may also be determined in accordance with manufacturer's instructions for the resin. Following elution 106, a harvest 108 operation is initiated by flowing the resin and dissociated biologic product through the filter module and collecting product from the permeate stream. Harvest continues via diafiltration through the filter module through a number of DVs as needed to reach a desired product yield. The harvest operation is preferably performed at an average permeate flow of about 500-4000 LMH, or about 1000-4000 LMH.

In some aspects, an additional filtration step utilizing a 0.8/0.2 micron or other similar graded membrane filter such as 0.45/0.2 micron glass fiber or polymeric capsule filter may be added to the eluent coming off the filter into the harvest vessel to remove any large particles that may have come off the TFF filter during the elution step.

It should also be understood that multiple harvest/capture flowpaths and modules as described herein may be connected in parallel. In an aspect, at least two flowpaths and modules are connected in parallel such that the resin of a first module may be regenerated while that in a second parallel flowpath/module continues to perform the capture and elution steps, thereby enabling continuous operation.

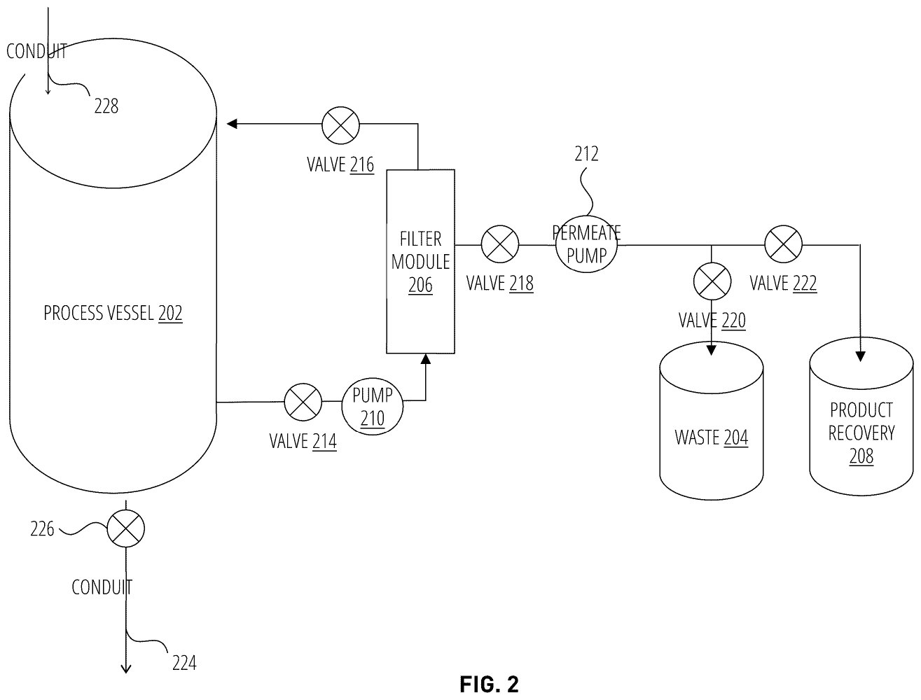

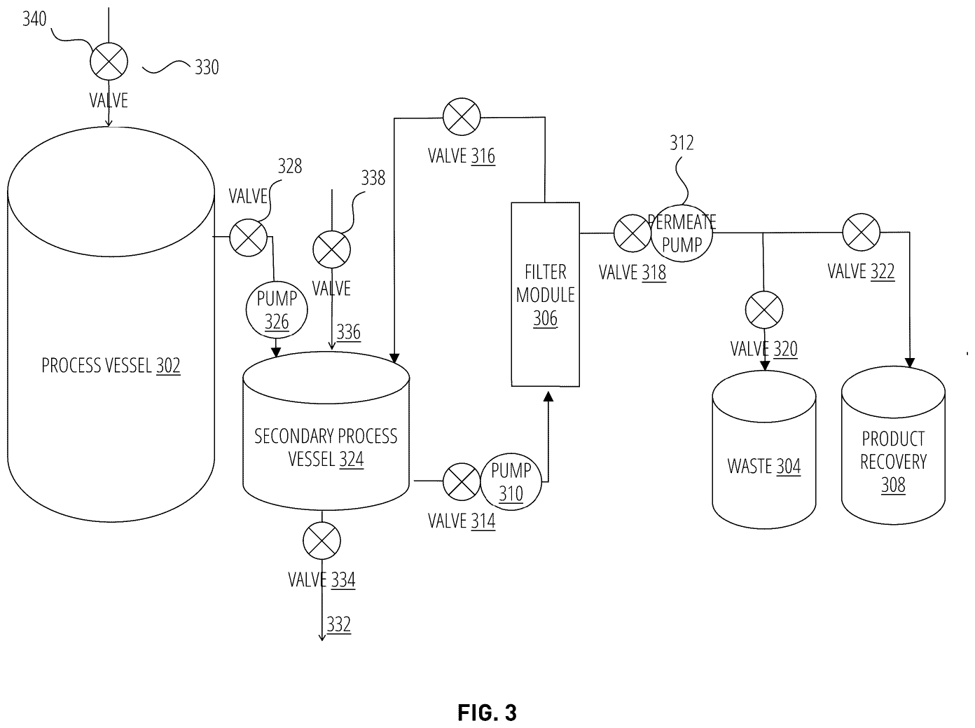

FIG. 2 schematically illustrates a system for performing a biologic product capture/harvest operation in accordance with an aspect of the present invention. As illustrated in FIG. 2, a unique and advantageous feature of the systems and methods described here is the use of a single fluid circuit interconnecting the process vessel 202 and the filter module 206 to perform the capture/harvest operation, rather than the use of multiple fluid circuits to perform these operations. The capture 102 step referred to in FIG. 1 is carried out in a process vessel 202, which may be a primary process vessel, such as a bioreacter as shown in FIG. 2, or a secondary process vessel 324 as shown in FIG. 3. Returning to FIG. 2, the figure illustrates a fluid conduit 228 of the process vessel 202 which may be used to add resin or process fluid to the process vessel in accordance with various methods described here. For example, where the capture 102 step is performed by direct addition of resin to a primary process vessel, such as a bioreactor, the conduit may be used to transfer resin to the primary process vessel.

Alternatively, where the resin is contained in a secondary process vessel, as illustrated in FIG. 3, the conduit may be used to transfer process fluid into the secondary vessel.

During capture, the process fluid is incubated with resin for a period of time under conditions suitable for binding of the biologic product to the resin, as discussed above. During this period, the process fluid and resin may be mixed using any suitable means. In one aspect of the methods described here, the mixing is performed by operation of an impeller of the process vessel 202. In accordance with this aspect, valve 226, valve 214 and valve 216 remain closed. In another aspect, the process fluid and resin may be mixed by recirculation through the filter module 206 by operation of pump 210. In accordance with this aspect, valve 214 and valve 216 are opened while valve 218 remains closed.

Following capture 102, a wash process in initiated. The wash operation may include a series of concentration and diafiltration steps as necessary to remove contaminants and impurities from the product-bound resin. During wash, valve 214, valve 216, valve 218, and valve 220 are opened, while valve 222 and valve 226 remain closed. Fluid circulates through the filter module 206 by operation of a retentate pump 210. The retentate pump may be, for example, a low shear centrifugal pump. The filter module 206 separates the fluid into a retentate stream containing product-bound resin and a permeate stream containing cells and/or cell lysate and other debris. The permeate stream may directed by operation of a permeate pump 212 to a waste 204 container, or in an alternative not depicted in the figure, the permeate stream may be directed to a secondary container, for example to capture producer cells. The permeate pump 212 may be, for example, a peristaltic pump. The retentate stream flows through a single retentate fluid circuit back into the process vessel 202. Buffer, cell culture media, or optionally additional process fluid as described below, may be added to the process vessel 202, for example via a conduit 228, in order to replace fluid volume lost to the permeate stream.

Following wash, the biologic product is eluted from the resin in the process vessel. During elution, the fluid in the process vessel is isolated by closing valve 218 and valve 220 and turning off the permeate pump 212. An elution buffer is added to the process vessel 202, for example via a conduit 228, and product-bound resin is incubated for a period of time to provide maximum dissociation of the biologic product from the resin. During elution, the mixture of resin-bound product and buffer may be mixed using any suitable means, for example via operation of an impeller in the process vessel or by recirculation through filter module 206, as discussed above in connection with capture.

Following elution, valve 218 and valve 222 are opened, permeate pump 212 is turned on, and biologic product in the permeate stream is collected in a product recovery 208 vessel. As with wash, the recovery operation may include a series of concentration and diafiltration steps through the filter module 206 until a desired amount of product is collected. Diafiltration buffer may be added to the system, for example, through conduit 228.

In an optional step, capture resin can be regenerated within the process vessel 202 or collected into a separate collection vessel through a conduit 224 by opening a valve 226 and regenerated off-line. If regenerating within the process vessel 202, regeneration buffer may be added through conduit 228. Regeneration of the resin may be accomplished based on the manufacturer's instructions.

The system may also comprise one or more of a flowmeter, a pressure sensor, and a controller.

The method for performing a biologic product capture/harvest operation in accordance with the present invention may be carried out in various ways. For example, in one aspect, a “constant feed concentration” or “CFC” operation may be utilized. In accordance with this aspect, capture is performed by direct addition of resin to a primary process vessel containing the process fluid, such as a bioreactor. As discussed above, and in accordance with all of the methods described here, the process fluid is characterized by high cell concentration and/or high turbidity. The resin is allowed to incubate with the process fluid for a period of time and under conditions suitable for maximum binding of cell product to the resin, optionally with mixing, as discussed above. Following capture, a portion of the fluid mixture is moved to a secondary vessel where a wash process is initiated under a “constant feed concentration” or “CFC” mode. In this mode of operation, the secondary vessel is kept at a constant volume by replacing fluid lost to the permeate stream with additional process fluid and resin mixture from the primary process vessel. The CFC operation continues until all of the process fluid mixture from the primary vessel has been added to the secondary vessel and concentrated to a desired volume. At this point, the contents of the secondary vessel are washed in a process that may include a combination of concentration and diafiltration operations until the resin is sufficiently free of contaminants and at a concentration suitable for elution.

Elution is initiated by addition of an elution buffer to the secondary vessel containing the resin-bound biologic product. Following incubation for a period of time and under conditions suitable for maximum elution of biologic product from the resin, as discussed above, a harvest operation is initiated and cell product is collected via diafiltration through the filter module. As discussed above, harvest may be performed in concentration, diafiltration, or a combination of both modes until the desired amount of product is collected. In an optional step, the capture resin may be regenerated within the secondary vessel or collected into a separate collection vessel and regenerated off-line.

FIG. 3 schematically illustrates a system for performing a biologic product capture/harvest operation in accordance with an aspect of the present invention utilizing a CFC operation. In accordance with this aspect, capture is initiated by adding capture resin directly to a primary process vessel 302 containing process fluid, such as a bioreactor, via a conduit 330. As discussed above, the resin and process fluid mixture is incubated for a period of time and under conditions suitable for maximum binding of cell product to the resin, optionally with mixing.

Following capture, a portion of the mixture from the primary process vessel 302 is transferred to a secondary process vessel 324, e.g., by opening a valve 328, which transfer may be facilitated by operation of a pump 326. Valves 340, 338, 334, 314, and 316 remain closed during this initial transfer process. Once the initial transfer is complete, a wash process is initiated via diafiltration through the filter module 306. The valve 328 between the primary and secondary process vessels is closed and valves 314 and 316 between the secondary process vessel 324 and the filter module 306 are opened, as well as valves 318 and 320 between the permeate outflow of the filter module and the permeate waste 304 container. The valve 322 to the product recovery 308 container remains closed during wash. Fluid recirculation between the secondary process vessel 324 and the filter module 306 is accomplished by the action of pump 310 while permeate outflow is directed to the permeate waste 304 container by operation of a permeate pump 312. During this process, fluid in the secondary process vessel 324 is maintained at a constant volume by adjusting a rate of fluid inflow from the primary process vessel 302 into the secondary process vessel 324 by operation of the pump 326. The rate of fluid inflow is adjusted to match the rate of permeate fluid outflow from the system, for example by operation of a fluid control mechanism.

Once all of the mixture of process fluid and resin has been added from the primary process vessel to the secondary process vessel, valve 328 may again be closed and pump 326 turned off while fluid continues to recirculate through the filter module 306, thereby concentrating the fluid in the secondary process vessel 324 due to fluid loss to the permeate stream.

Once the mixture of process fluid and resin has been sufficiently concentrated in the secondary process vessel, a further wash process is initiated by addition of diafiltration buffer to the secondary process vessel, for example by opening valve 338 and allowing buffer to flow into the secondary process vessel via a conduit 336. wash proceeds via a process that may include a combination of concentration and diafiltration operations through the filter module 306 until the resin is sufficiently free of contaminants and at a concentration suitable for elution.

For elution, the fluid loop is closed, for example by closing all valves except valves 314 and 316 and elution is initiated by addition of an elution buffer to the secondary process vessel 324, for example by opening valve 338 and allowing buffer into the system via a conduit 336. Once a sufficient volume of buffer has been added, valve 338 is closed and the mixture is incubated for a period of time under conditions suitable for maximum elution of the biologic product from the resin, optionally with mixing as discussed above.

Following elution, a harvest operation is initiated by opening valves 318 and 322 (valve 320 remains closed) and recirculating fluid through the filter module 306 by operation of pump 310. As discussed above, biologic product is recovered in the permeate stream by diafiltration through the filter module 206 and collected in a product recovery 308 vessel. Also as discussed above, harvest may be performed using a series of diafiltration and concentration processes until a desired amount of product is collected. In an optional step, the capture resin may be regenerated within the secondary vessel or collected into a separate collection vessel, for example by opening a valve 334 and flushing the resin from the secondary vessel via a conduit 332 where it may be collected and regenerated off-line. Where the resin is regenerated in the secondary vessel, a regeneration buffer may be added to the vessel, e.g., via conduit 336 or other suitable conduit or port.

It will be appreciated that the system depicted in FIG. 3 may also be utilized in a batch process where discrete volumes or “batches” of process fluid are separately subjected to capture and wash. For example, in an exemplary batch process, valve 328 is opened to allow a first volume of process fluid from the primary process vessel 302 to enter a secondary process vessel 324 containing the capture resin, for example via operation of pump 326. Capture is performed as described above. Following capture, a wash process is initiated as described above except there is no continuous feed from the primary process vessel 302 into the secondary process vessel 324, as in the CFC operation. Instead, valve 328 remains closed until the first volume of process fluid and resin has been sufficiently washed via diafiltration through the filter module 306, and concentrated. Once this is accomplished, valve 328 is opened and a second volume of process fluid is added to the resin in the secondary process vessel 302. Capture and wash are repeated in the same manner as described for the first volume of process fluid. Capture and wash are repeated in batch mode until all of the fluid from the primary vessel 302 has been added to the secondary vessel 324 and processed. Elution, harvest, and optional regeneration of the capture resin are carried out as described above.

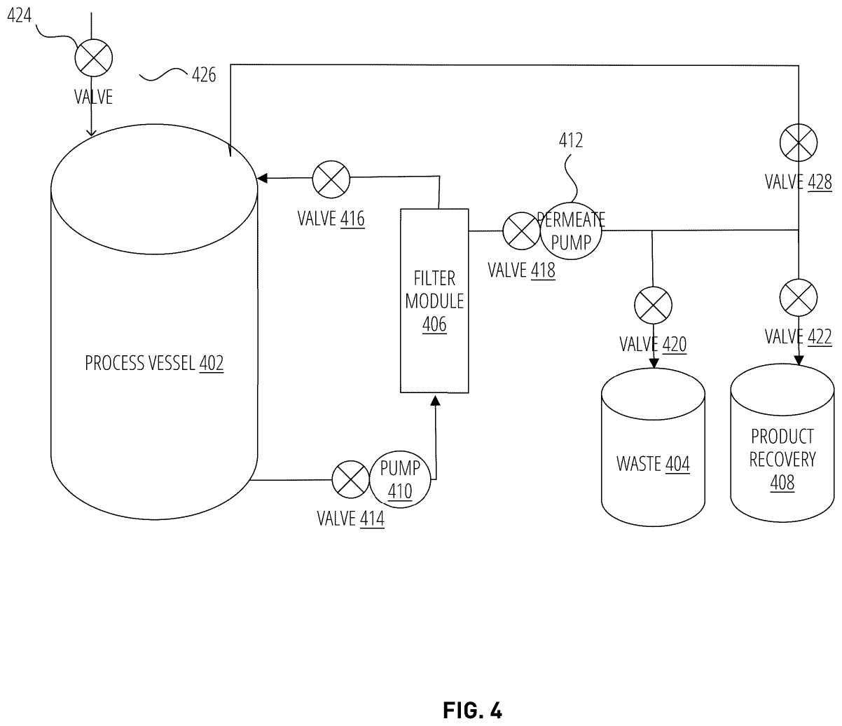

In another aspect, the capture/harvest operation may be performed using “dead-end” capture. FIG. 4. illustrates a system for use in a “dead-end” capture operation. In accordance with this aspect, capture is initiated in the same manner as described above, that is by direct contact of process fluid and a capture resin, for example by addition of resin directly to a primary process vessel containing process fluid, or alternatively by addition of a volume of process fluid to secondary process vessel containing resin. Unlike the methods discussed above, here valve 416 is closed to block the retentate stream and a fluid circuit between the process vessel 402 and the filter module 406 operating in a “dead-end” mode is created through the permeate stream by opening valves 414, 418, and 428. Permeate fluid is thereby collected and returned to the process vessel, as valves 420 and 422 also remain closed. Following capture, valves 416 and 420 are opened while valve 428 is closed and valve 422 remains closed to create a fluid circuit between the process vessel 402 and the filter module 406 through the retentate fluid stream. wash proceeds as discussed above by diafiltration of the product-bound resin through the filter module 406, with process fluid containing the resin remaining in the retentate stream and being recirculated through the module while permeate fluid is removed from the system and collected in a waste container. Elution, harvest, and optional regeneration of the resin are performed as described above.

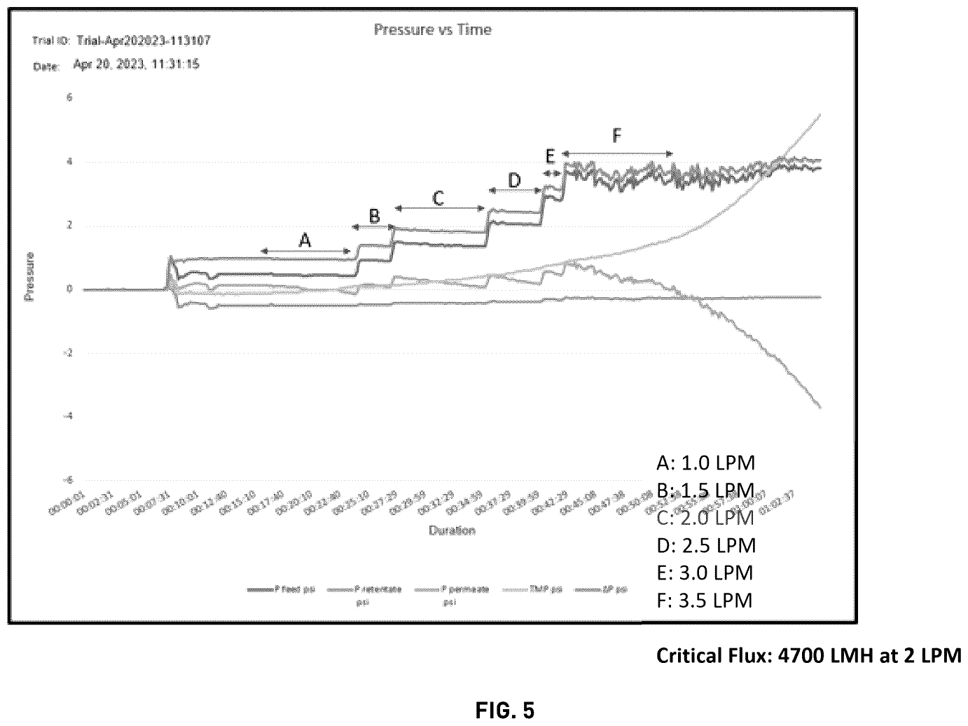

FIG. 5 is a line graph of pressure (psi) versus time (minutes) showing a critical process flux of 4700 LMH at point C, 2 liters per minute (LPM). Critical process flux provides the maximum operating flux for a given feed flow using a representative feed stream, for example a process fluid comprising cell culture fluid and/or cell lysate with resin, or other non-water feed stream. In practice, the critical process flux will be significantly lower than the NWP of the membrane and must be determined experimentally. Here, we analyzed using different feed flow rates in order to determine a suitable range for operating flux, which was determined here to be in a range of from about 500-7000 LMH.

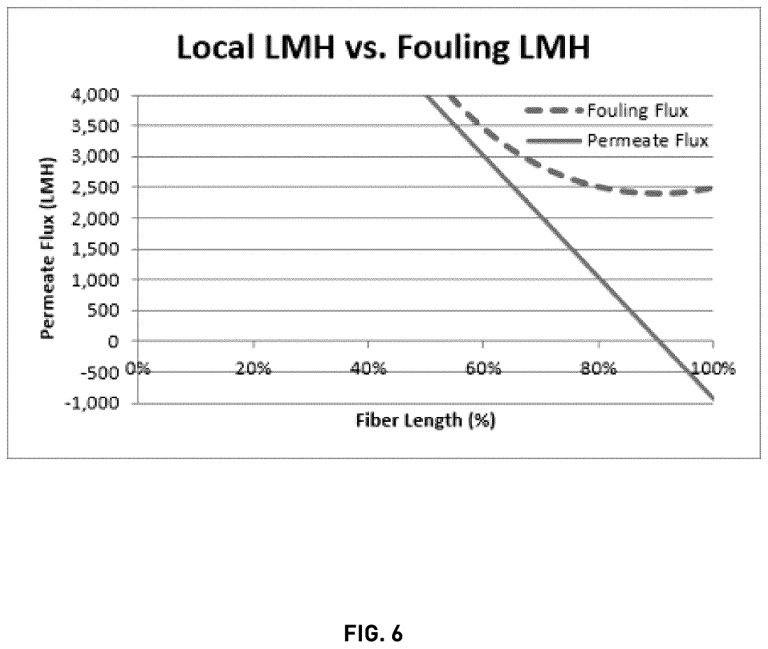

FIG. 6 shows results of a model indicating that a 67% porous TFDF membrane would not foul at an average permeate flow of 4,000 LMH. The model predictions were tested as described in the following experiments.

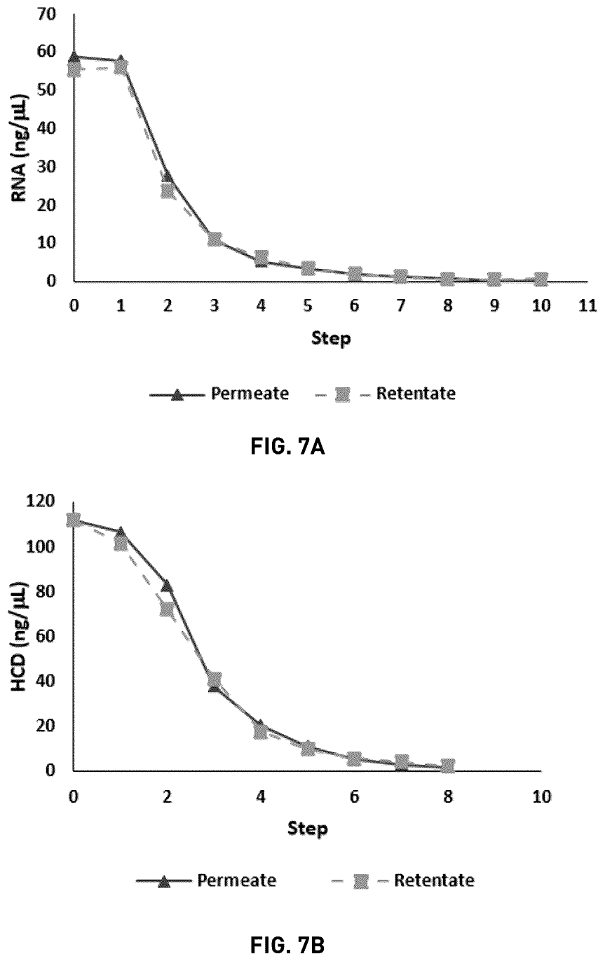

FIG. 7A illustrates performance of a 67% porous TFDF membrane in clearing RNA from cell lysate. Initial cell density was 6.7E6 cells per ml, prior to lysis. This experiment shows 100% clearance of RNA. Both the retentate and permeate streams showed an equal and steady decline of Rna during the process (2× initial concentration, step 1; 9× diavolume wash, step 2-10). The data presented above suggests that RNA clearance of the source liquid may be achieved with minimal, or no need of enzymatic degradation using endonucleases, such as Benzonase® or Decontaminase®.

FIG. 7B illustrates performance of a 67% porous TFDF membrane in clearing host cell DNA (HCD) from a mixture of cell lysate and affinity resin (1% AviPure). Initial cell density was 9E6 cells per ml, prior to lysis. This experiment shows approximately 100% clearance of DNA. Both the retentate and permeate streams showed an equal and steady decline of HCD during the process (2× initial concentration, step 1; 7× diavolume wash, step 2-8). The data presented above suggests that HCD clearance of the source liquid may be achieved with minimal, or no need of enzymatic degradation using endonucleases, such as Benzonase® or Decontaminase®.

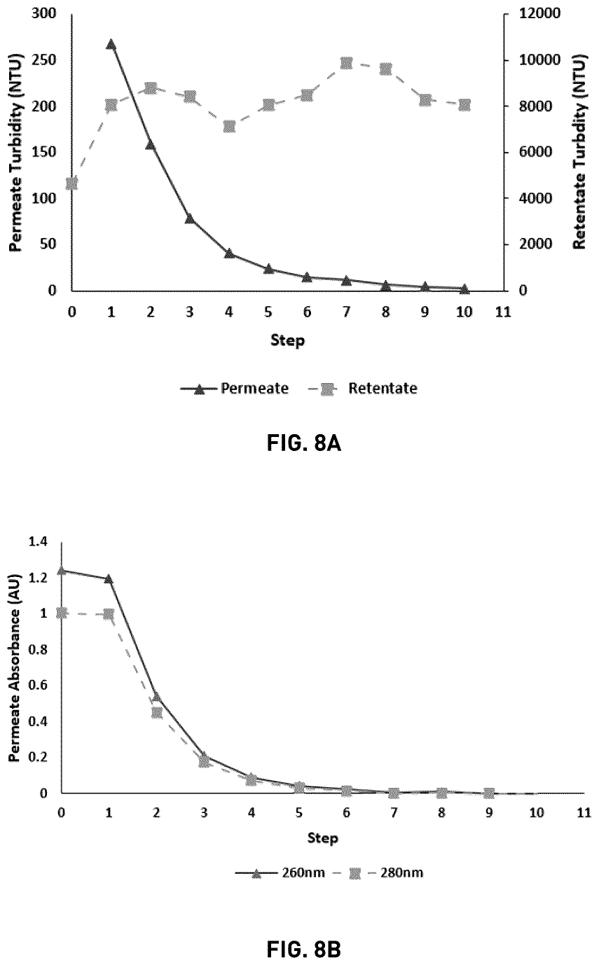

FIG. 8A illustrates performance of a 67% porous TFDF membrane in reducing turbidity from a mixture of cell lysate and affinity resin. Initial cell density was 6.7E6 cells per ml, prior to lysis. The initial turbidity of the cell lysate was 509 NTU. Addition of affinity resin to the lysate increased turbidity to 4670 NTU, suggesting that resin alone was responsible for about 4000 NTU. Following completion of an initial 2× concentration cycle of the process fluid and resin mixture through the TFDF module step, step 1 in the graph, turbidity of the retentate stream increased from about 4000 to about 8000 NTU, indicating that resin was highly retained by the TFDF module. High resin retention is further evidenced by the fact that the turbidity of the retentate remained at about 8000 NTU during the following diafiltration steps. Additionally, permeate turbidity steadily decreased from 509 NTU to about 260 NTU in the first concentration step, suggesting that turbidity from cell lysate was readily passing through the filter. Turbidity values steadily decreased during the 9 DVs, suggesting that cell lysate was not being retained and passed readily through the 67% porous TFDF tube.

FIG. 8B illustrates performance of a 67% porous TFDF membrane in clearance of cell lysate. Initial cell density was 6.7E6 cells per ml, prior to lysis. The clearance of cell lysate was measure by UV-Vis at both 260 nm and 280 nm before and after addition of affinity resin. Affinity resin absorbs at 975 nm and had minimal impact on A₂₆₀ and A₂₈₀ values (data not shown). Since there was no AAV present in this experiment, a decrease in A₂₆₀ and A₂₈₀ values signify clearance of lysate constituents only, such as host cell proteins, DNA and RNA. No change in absorbance, or a slight decrease, after the first concentration step (2× CF), indicates that cell lysate is freely passing through the 67% porous TFDF tube, with minimal retention. If any retention in cell lysate components were to occur during the concentration step, an increase in signal would be observed. This was further confirmed as the absorbance values continued to decrease throughout diafiltrations steps, indicating high passage of cell lysate through 67% porous TFDF tubes.

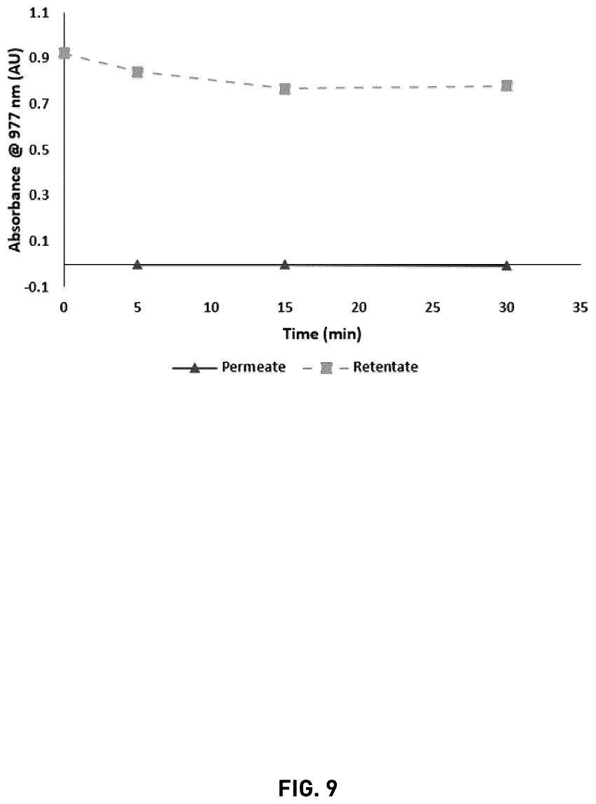

FIG. 9 illustrates the high level of resin retention by the 67% porous TFDF membrane. The retention of Poros 50HQ Resin (50 μm average diameter) was tested in 1×PBS on 67% porous TFDF tubes in full recirculation mode. The feed flow rate was set to 800 mL/min (˜1400 s⁻¹) and the permeate pump was set to 100 mL/min (2000 LMH). The data collected indicates that the 67% porous TFDF tube retained all of the resin during the 30-minute experiment, as no significant absorbance values were detected in the permeate. After 30 minutes of full recirculation, the permeate pump was turned off and two readings of the retentate were measured, providing a yield of 91% for both.

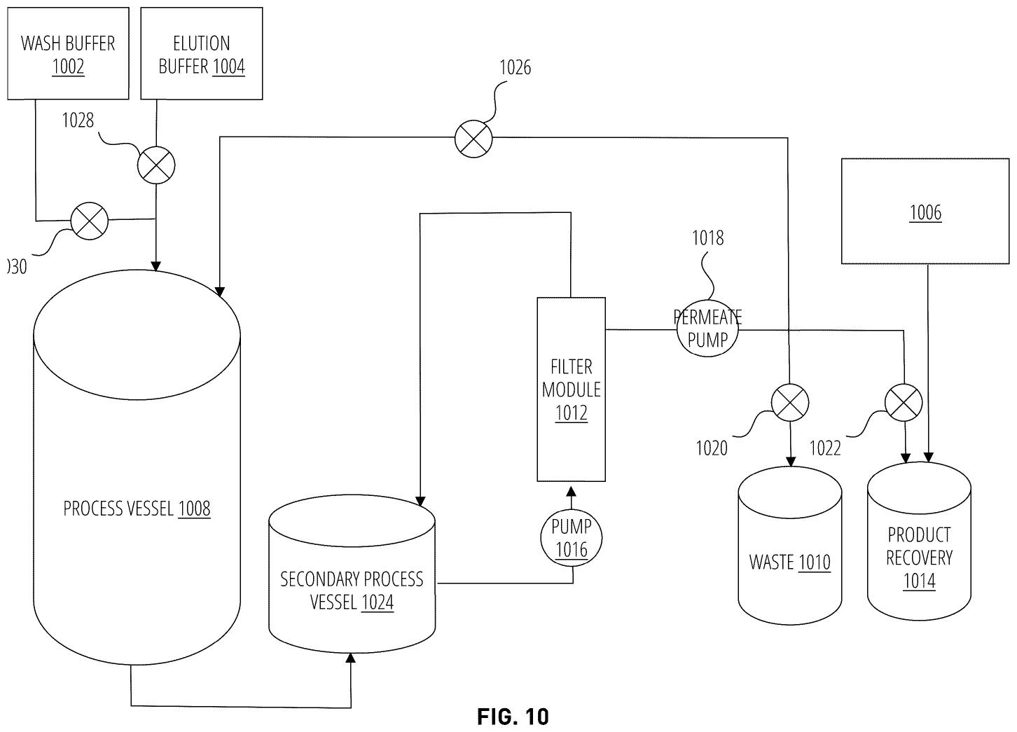

FIG. 10 illustrates a “constant feed concentration” system in accordance with an aspect of the invention. This system was utilized in a proof of concept experiment where the biologic product was AAV virus particles (vp). Briefly, the capture operation was initiated by adding approximately 12 mL of affinity resin to 1-L of lysed HEK293 cells containing 3.4E10 vp/mL of AAV9 in the process vessel 1008. The resin was allowed to incubate in the process vessel 1008 for 2 hours before being transferred to a 200 mL secondary process vessel 1024 via peristaltic pump (pump not shown) for an additional 1 hour of incubation and mixing via recirculation through the filter module 1012. During this additional 1 hour incubation, which may be referred to as “full recirculation mode” the permeate is returned to the process vessel 1008 and returned permeate material is continuously fed into the secondary process vessel 1024 to ensure maximum binding with the resin. At the same time, the affinity resin is retained in the retentate flow of the filter module 1012 in a recirculating fluid loop and is not returned to the process vessel 1008. Following capture, a concentration and wash process was initiated. First, the resin/lysate mixture was concentrated to ˜200 mL by operating the filter module 1012 in CFC mode. Then wash was performed by diafiltration through the filter module 1012 with 8 DVs of wash buffer (phosphate buffered saline, or PBS). Following diafiltration, the 200 mL of working volume was concentrated to ˜ 150 mL in a final concentration step to accommodate the volume of elution buffer. For elution, 50 mL of elution buffer (100 mM Glycine, 150 mM NaCl, 0.01% P-188, pH 2) was added directly to the secondary process vessel 1024 (not shown in diagram) via peristaltic pump (<1 min addition time). Diafiltration with elution buffer was started immediately using vacuum. Eluted virus particles were collected in a product recovery 1014 vessel preloaded with ˜170 mL (10% of final volume) neutralization buffer (1 M Tris Base, pH 9) so that freshly cluted virus was pH balanced to PH ˜7. The cluted virus was washed with 8 DVs of elution buffer, providing a final volume of approximately 1.7-L.

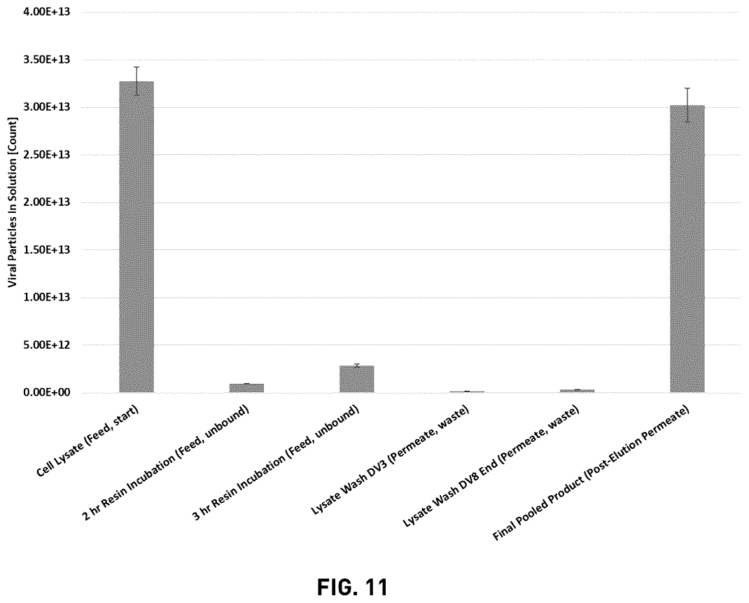

Results are shown in FIG. 11. The initial counts of virus particles within the 1-L primary process vessel were determined to be 3.40E13 via ELISA, shown in first bar from left labeled “cell lysate (feed, start)”. Following the initial two hours of capture in the primary process vessel in which affinity resin was added directly to cell lysate, only 3% of unbound virus particles were found in solution, indicating high binding of virus particles to the resin. After an additional 1 hour of binding in full recirculation mode through the filter module, as described above, the amount of virus particles in solution increased to 8%. Minimal virus particles were found in the wash fluid after 3 and 8 DVs during wash, indicating strong binding of virus particles to the resin. Following elution and washing through 8 DV of elution buffer as described above, virus particles in the product recovery vessel were quantified by ELISA. As illustrated by the last bar from left labeled “final pooled product (post-elution permeate)” over 3.00E13 virus particles were recovered, demonstrating a 92% overall recovery achieved by this method of harvest and capture of virus particles directly from crude cell lysate, without any prior wash steps.

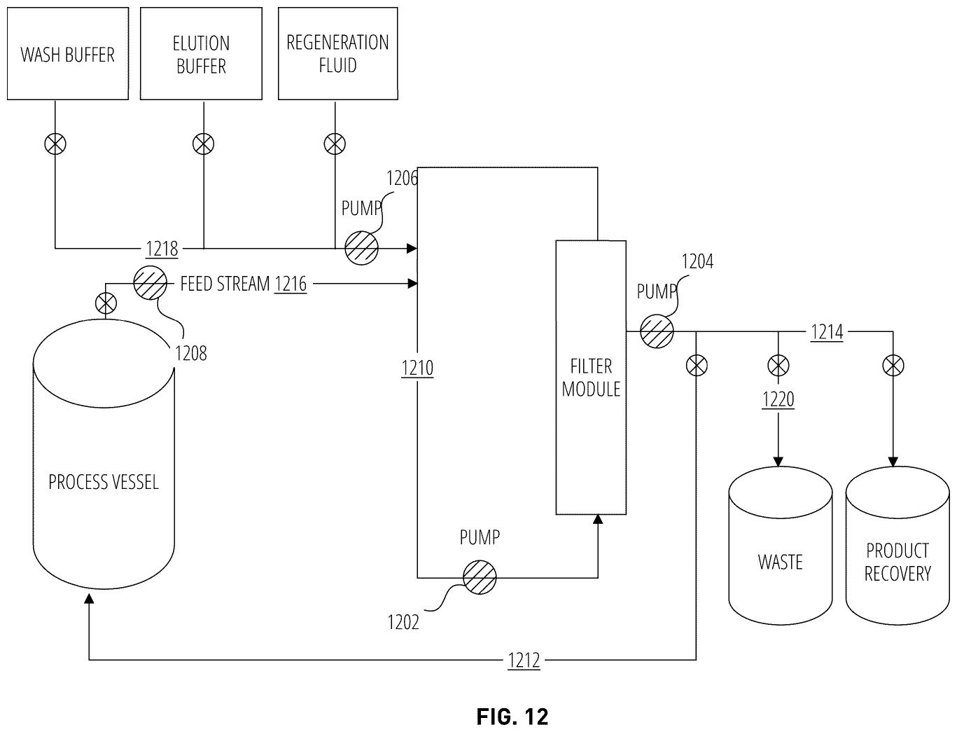

Another configuration of a system in accordance with the present invention is shown in FIG. 12. In this configuration, the resin recirculates within the filter module flow path 1210, which may also be referred to as the “retentate fluid circuit”. Capture is performed by introducing cell culture fluid or lysate to the fluid circuit via a conduit 1216 and recirculating with the resin for a period of time to allow for binding of the target biologic to the resin. In this capture step, both the retentate and permeate fluid streams recirculate in the fluid circuit. Following capture, a concentration and wash process is initiated. During this process wash buffer along with cells, debris, and cell culture fluid or lysate in the permeate are passed directly to waste while the biologic product-bound resin is retained within the retentate fluid circuit 1210. A concentration step may be performed before and/or after the wash process. Following the concentration and wash processes, an elution process is performed to release the biologic product from the resin by introducing an elution buffer to the fluid circuit 1210, e.g., via a conduit 1218. Biologic product, now in the permeate 1214, is collected in a product recovery vessel.

In an aspect, the filter module includes a large porosity filter medium constructed of non-woven fibers having pore size in the range of 50-200 microns. In aspects, the filter medium may be in the form of a tubular/spiral flat sheet, which may also be referred to herein as a tangential flow chromatography filter or “TFCF”. In aspects, the TFCF is constructed from a nonwoven polypropylene/polyethylene polymer having a pore size of from 50-200 microns formed into a tubular membrane, for example by spiral winding of a flat sheet TFCF membrane. Suitable membranes include nonwoven wetlaid membranes. In aspects, the TFCF medium is not formed by extrusion.

In aspects, a plurality of filter modules may be included in series or in parallel. In aspects where the biologic product is contained in a lysate, an acid pretreatment of the lysate may be performed prior to introduction into the retentate fluid circuit. In some aspects, a further optional filtration may be performed on the permeate containing the biologic product following elution from the resin.

As depicted in the figure, there is no recirculation reservoir. However, a recirculation reservoir may be included for the resin slurry at larger scales.

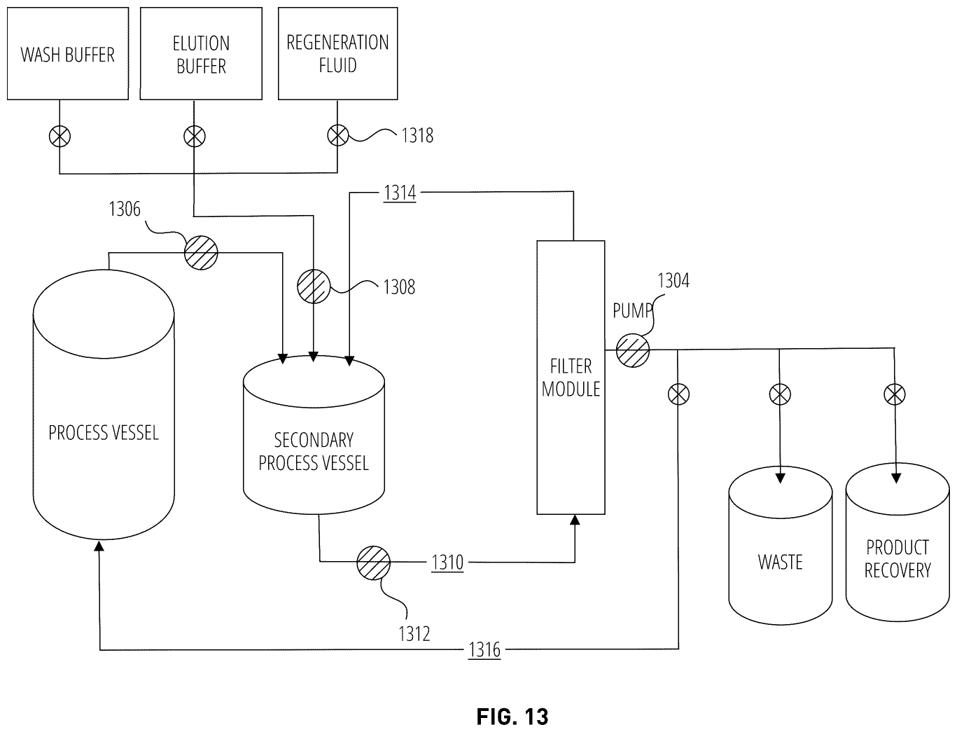

FIG. 13 depicts an alternate configuration of the system in FIG. 12 where a secondary process vessel is included in the fluid circuit 1314 to receive cell culture fluid or lysate from a process vessel, as well as wash buffer, elution buffer, and regeneration fluid from external vessels at the appropriate points in the harvest-capture process.

In an aspect, the filter module includes a large porosity filter. In aspects, a plurality of filter modules may be included in series or in parallel. In aspects where the biologic product is contained in a lysate, an acid pretreatment of the lysate may be performed prior to introduction into the retentate fluid circuit. In some aspects, a further optional filtration may be performed on the permeate containing the biologic product following elution from the resin.

A system in accordance with FIG. 12 was tested using an IgG-binding Protein A resin and two AAV resins. In initial experiments, the binding affinity of each resin for its target ligand was evaluated using batch adsorptions studies, detailed in the following two paragraphs.

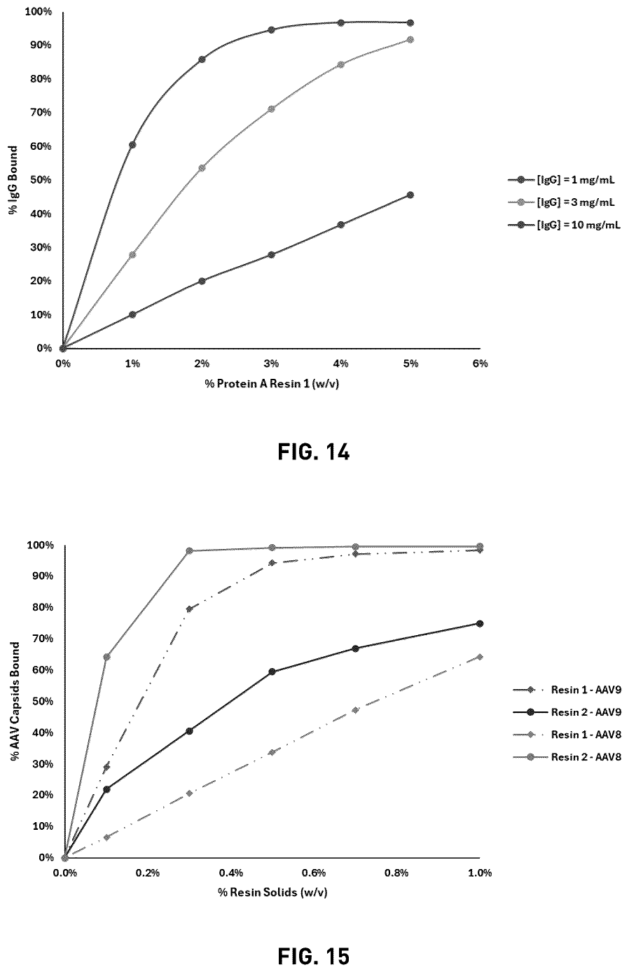

As shown in FIG. 14, IgG-binding affinity of a Protein A resin was measured via batch adsorption studies. Briefly, aliquots of Protein-A resin (% Protein A Resin was varied between 1-5% w/v) were incubated with solutions of IgG in PBS in a range of concentration between 1 to 10 mg/mL for 1 hour followed by centrifugation and collection of supernatant. The amount of IgG adsorbed on each resin aliquot was calculated based on the equilibrium concentration of IgG in the supernatant. For 3% resin and 1 mg/mL IgG, more than 85% of IgG was bound at 30 minutes and more than 90% was bound at 60 minutes. The resin load was about 70 mg IgG/ml resin.

The AAV-binding affinity of Resin 1 and Resin 2 for AAV8 and AAV9 was measured via batch adsorption studies. Briefly, aliquots of resin (% Resin was varied between 1-5% w/v) were incubated with AAV8 or AAV9 at a titer of about 1E12 virus particles (vp)/ml for 3 hours followed by centrifugation and collection of supernatant. The amount of AAV adsorbed on each resin aliquot was calculated based on the equilibrium concentration of AAV in the supernatant. As shown in FIG. 15, the data indicate distinct binding preferences between different serotypes and resins. The resin load was between 1E14 and 1E15 vp/ml resin.

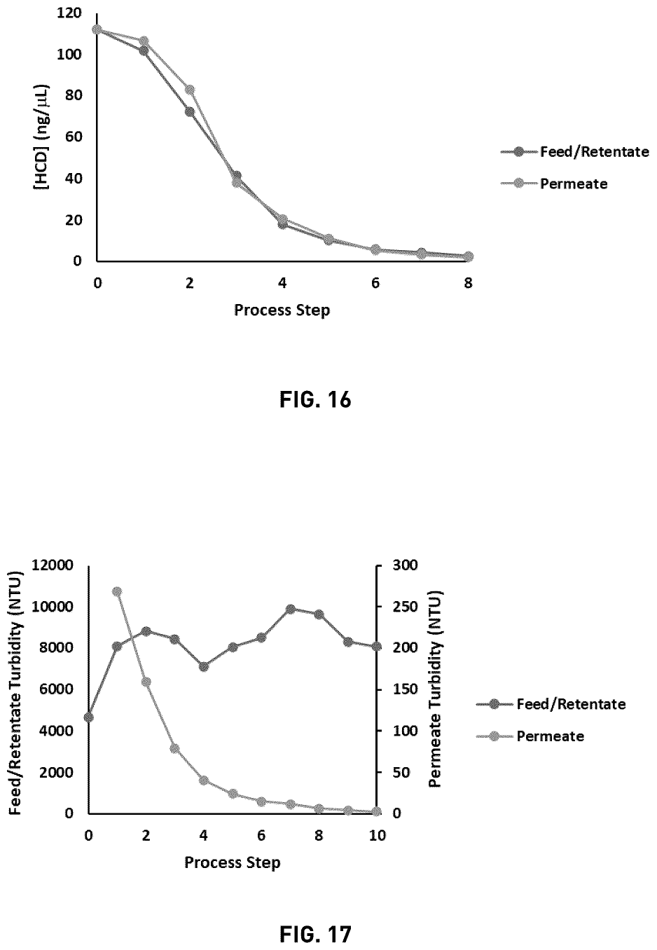

FIG. 16 shows host cell DNA (HCD) reduction in both the feed/retentate line and the permeate utilizing a system in accordance with FIG. 12. HCD was initially almost 120 ng/ul and was reduced to almost zero following a concentration and 2-8 diafiltration volumes (DVs). The data show a very high clearance of HCD of about 98%.

FIG. 17 shows reduction in turbidity in the permeate (light line) compared to the feed/retentate (upper, darker line), through a 2-fold concentration followed by 1-10 diafiltration volumes (DVs) utilizing a system in accordance with FIG. 12. The data show about 99% turbidity reduction in the permeate. The retentate remains at a turbidity of about 8000 NTU due to the presence of the resin which remains in the retentate recirculation loop.

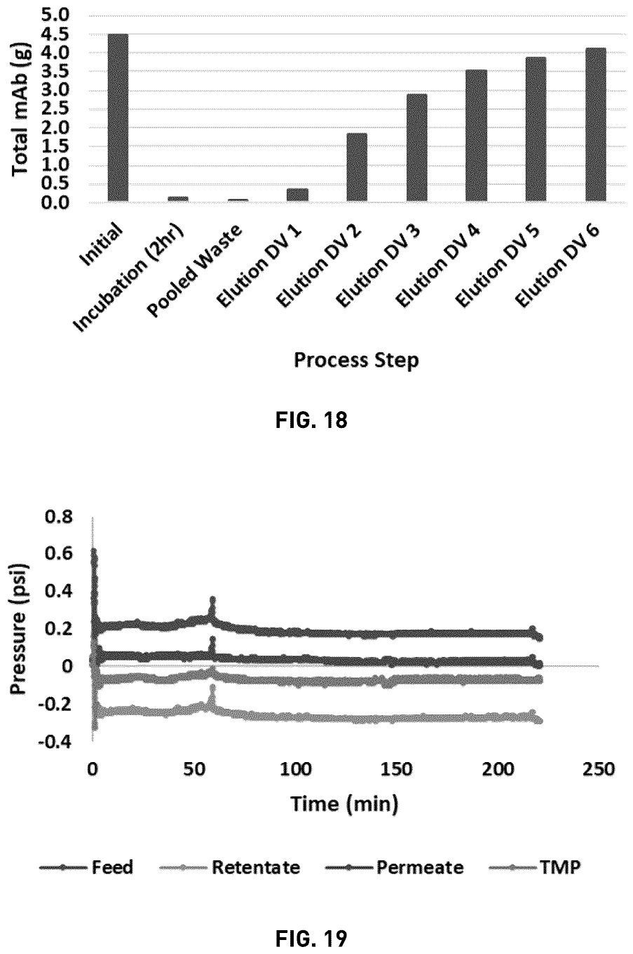

FIG. 18 shows product recovery from a 1-liter harvest-capture scale down for monoclonal antibody (mAb) harvest from Chinese Hamster Ovary (CHO) cells utilizing a system in accordance with FIG. 12. CHO cells were at 50E6 cells/ml and the concentration of mAb was 4.4 mg/ml. Protein A Resin 1 was present at 5% w/v, the filter surface area was 4.3 cm² and the permeate flux was 2,000 LMH. Antibody capture at 60 minutes was greater than 90% (total incubation was 120 minutes). The final yield was about 92%.

FIG. 19 shows the pressure profiles for the 1-liter mAb harvest-capture scale down in FIG. 18 utilizing a system in accordance with FIG. 12. The data show no change to process pressures during entire run which indicates consistency in flux and no filter fouling.

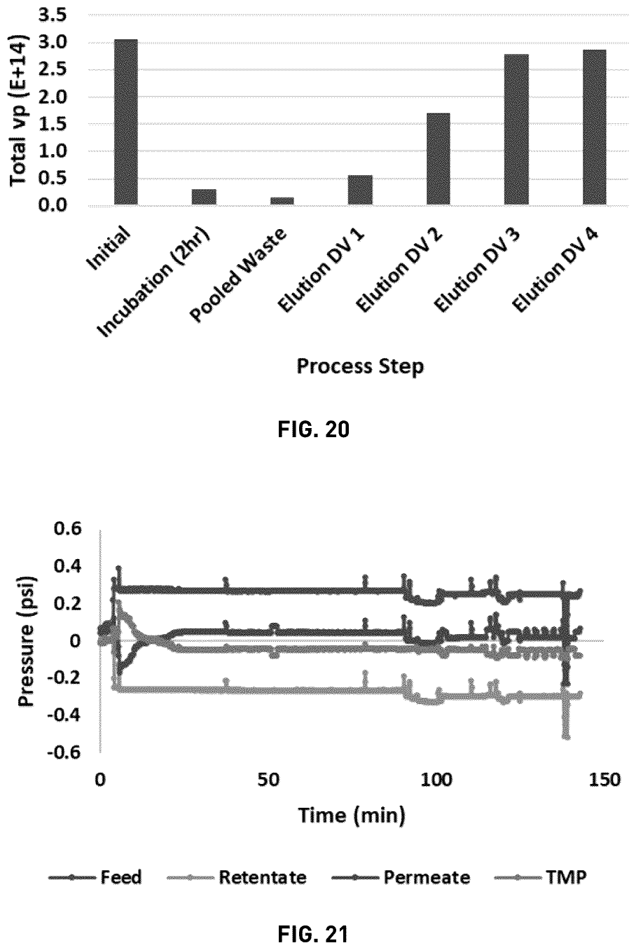

FIG. 20 shows product recovery from a 1-liter harvest-capture scale down for AAV9 harvest from HEK293 cells utilizing a system in accordance with FIG. 13. HEK293 cells were at a concentration of 10E6 cells/ml and viral capsid titer was about 3E11 vp/ml. AAV9 Resin 1 was present at 0.5% w/v, the filter surface area was 3.0 cm² and the permeate flux was 2,000 LMH. AAV capture at 120 minutes was greater than 95% (total incubation was 120 minutes). The final yield was about 90%.

FIG. 21 shows pressure profiles for the 1-liter AAV harvest-capture scale down in FIG. 20. The data show no change to process pressures during entire run which indicates consistency in flux and no filter fouling.

According to some aspects of the methods described here, the capture step may be performed in one of two ways. In the first, capture is performed by addition of resin to a primary process vessel or a secondary process vessel, which may also be referred to as a “feed vessel”. This method may be referred to herein as “batch capture”. In the second, capture is performed by preloading the resin into a retentate fluid circuit 1210 of the filtration module, which may also be referred to as a “retentate recirculation loop” or a “fluidized bed”. This method may be referred to herein as “inline capture”. During the inline capture process, the resin slurry volume inside the flowpath is maintained at a constant volume. This is accomplished by maintaining the same flowrate for both the feed stream 1216 (which has a high concentration of product) and the permeate stream (which has a low product concentration) leaving the flowpath. Experiments were performed to compare efficiency of capture for the batch and inline methods utilizing monoclonal antibody and AAV as the target biologic product. In the inline capture process, the permeate was recycled back into the feed vessel. This is the ideal method for capture with respect to waste handling but may increase process time due to feed dilution by the returning low concentration waste stream.

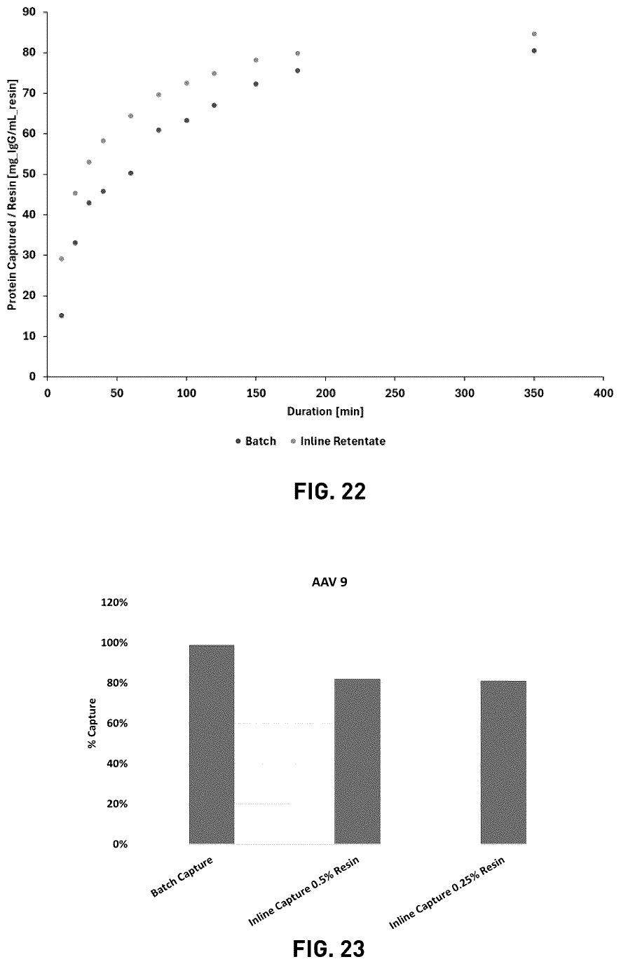

FIG. 22 shows capture of monoclonal antibody (mAb) as mg IgG per ml resin over time (minutes). The top curve represents the inline capture and the bottom curve represents the batch capture. In this experiment, the inline capture process was more efficient and reached a greater saturation compared to batch capture. Experiments were performed using 200 mL Chinese Hamster Ovary (CHO) cell supernatant with an initial protein titer of 2.39 mg/mL. For each of batch and inline capture, 5 grams of Protein A affinity resin was used. This means that in the batch process, the resin is present at a lower concentration, 2.5% wt/vol (5 g resin/200 ml liquid), compared to the inline process where the resin slurry is maintained at a concentration of 40% wt/vol (5 g resin/12. 5 ml liquid). The Loop Feed/Bleed Rate for the inline process was 10 mL/min.

A second experiment was conducted with AAV9 as the target biologic product. In this experiment only a single, final data point was taken following three (3) hours of contact with the resin. Results are shown in FIG. 23. In this experiment, batch capture outperformed inline capture, possibly due to the fact that the starting feed volume was higher while the feed/bleed rate was maintained the same, at 10 mL/min. Thus, in this experiment unbound product had less opportunity to bind to the resin compared to the experiment discussed above in relation to FIG. 22. The data also show that the same amount of virus was captured inline at half the loading ratio of resin (0.5% vs 0.25%). This indicates that less resin was required to capture the same amount of virus using this method. However, this result may be due to failure to reach equilibrium after three hours.

Experiments were performed using 1000 mL of AAV 9 with an initial titer 2.32E11 vp/mL. Affinity resin was 5 g or 2.5 g of AVIPure AAV 9 resin. The loop feed/bleed rate was the same as the monoclonal antibody capture experiment, at 10 mL/min. Batch Capture, 0.5% solids, Inline Capture, 0.5% solids, 0.25% solids. Solids are v/v % of resin added relative to the starting liquid volume. Phase Ratio: 40% and 20%, where phase ratio is the ratio of liquid to solid slurry in the flowpath at the time of capture. This parameter is an indicator of how concentrated the resin is in the flowpath during inline capture. The main difference between the batch and inline methods is that resin is concentrated in the flowpath loop during inline capture while it is evenly dispersed in the feed container during batch capture.

In an alternative process, referred to as a “single pass process”, the permeate stream is not returned to the process vessel, but instead is sent directly to waste via 1220. For a single pass process, enough residence time is required for the resin to capture the target biologic, similar to a traditional chromatography column process.

Results of a non-optimized full single pass inline process for capture/harvest of AAV are shown in FIG. 24. With reference to FIG. 12, in a single pass process, the permeate stream is not returned to the process vessel, but instead is sent directly to waste via 1220. Initial viral titer is set to 100% (left most bar). The next six bars, labelled “Perm Inline Hour #” represent hourly measurements of viral product in the permeate stream following 1, 2, 3, 4, 5, or 6 hours of capture. The data show that the amount of product lost to waste is the same at each time point, indicating that resin binding sites are in excess permitting maximum binding with respect to the residence time. The column labeled “Waste” represents a pooled sample of all product waste from capture and wash steps. The final column, “Pooled Product” represents AAV recovery from the harvest operation. Overall, the data indicate the feasibility of the process. Yields can be improved, for example, by optimizing the elution buffer composition or resin saturation.

This experiment was performed utilizing 1 liter AAV9 lysate, a 5-minute residence time, a gentle feed stream, a loading ratio of 3.7E13 vp/ml resin. The residence time refers to the average time that any product particle will interact with the resin in the flowpath and is calculated as Liquid Volume in the fluid circuit divided by the permeate flow rate.

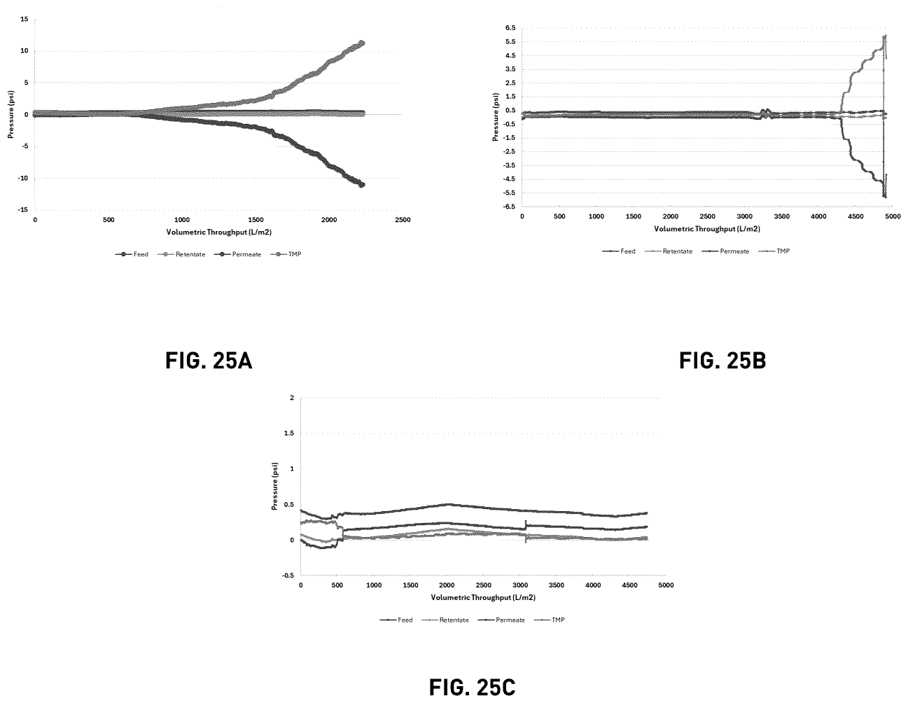

A further series of experiments was undertaken to study conditions under which filter fouling occurs and implement additional processes to reduce fouling. HEK cells were mechanically lysed on an orbital shaker in a buffer containing 50 mM Tris Base, 2 mM MgCl2, and 1% Polysorbate 20 (Tween 20). Following lysis, lysate was either (1) untreated (control), (2) treated with citric acid (20 minutes in buffer where the pH was lowered to 4.1 by addition of citric acid), or (3) treated with endonuclease at 50 units/ml. Batch capture was simulated with a 2 hour incubation at room temperature, in the absence of resin. Lysate was then subjected to single pass filtration through a 50 micron wet laid nonwoven polypropylene/polyethylene polymer filter at a recirculation rate of 500 ml/min and a flux of 2000 LMH. Membrane fouling was followed by plots of pressure (psi) versus volumetric output (L/m²). FIG. 25A shows the pressure profile for the control (no acid or endonuclease treatment of lysate). In the control, both the feed and retentate streams, which do not pass through the filter module, exhibit a constant pressure beyond an output of 2000 L/m². In contrast, both the permeate and trans membrane pressure (TMP) begin to deviate from baseline between 1000 and 1500 L/m², indicating inconsistencies in flux and membrane fouling. These data indicate that the standard HEK lysis procedure generates large cell debris that will not pass through the 50 um filter and will cause the filter to clog during concentration.

FIG. 25B shows the pressure profile for lysate subjected to low pH conditions by treatment with citric acid. Here, in contrast to the control run, there was no change in process pressures until beyond an output of 4000 L/m². These data indicate that the citric acid treatment prevented fouling during concentration, but fouling was observed during subsequent diafiltration.

FIG. 25C shows the pressure profile for lysate treated with endonuclease. Here, there was no change in process pressures for the entire experiment, which extended beyond an output of 4500 L/m². These data indicate that the endonuclease treatment prevented fouling during both concentration and diafiltration.

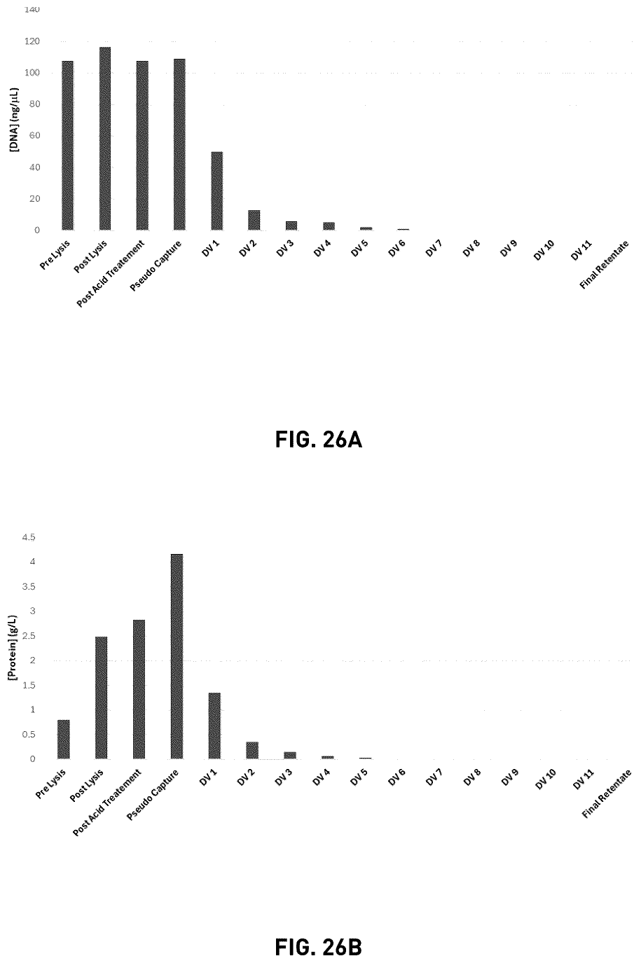

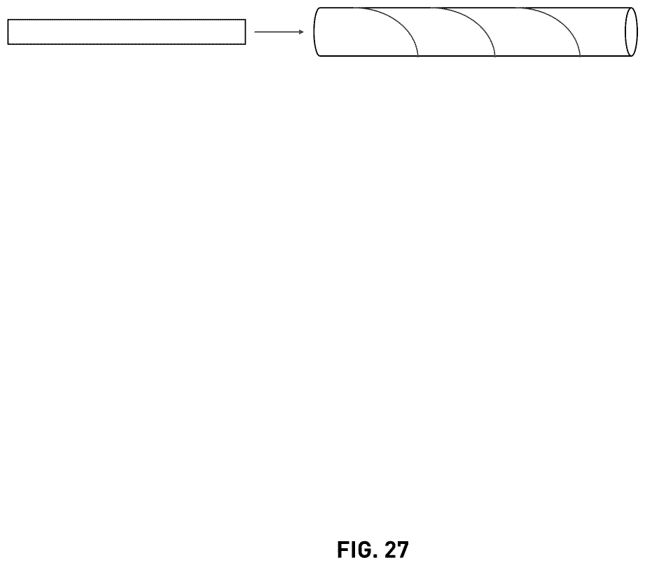

As shown in FIG. 26A and FIG. 26B, citric acid treatment also resulted in good clearance of DNA (FIG. 26A) and protein (FIG. 26B) during the diafiltration wash steps.

Additional embodiments are set forth below.

Embodiment 1: A method for isolating a biologic product from a process fluid characterized by high cell density and/or high turbidity, comprising performing a capture operation by contacting the process fluid with a capture resin in a retentate fluid circuit of a filtration module for a period of time sufficient to allow binding of the biologic product to the resin; performing a wash operation by separating the process fluid and resin mixture into permeate and retentate fluid streams of the filtration module and recirculating the retentate fluid stream through the retentate fluid circuit for a first number of diafiltration volumes (DVs) while directing the permeate stream to a waste container outside the fluid circuit, thereby producing a clarified retentate fluid, and optionally concentrating the clarified retentate fluid in the retentate fluid circuit; performing an elution operation by contacting the clarified retentate fluid with a volume of elution buffer in the retentate fluid circuit for a period of time sufficient to allow disassociation of the biologic product from the resin; performing a harvest operation by circulating the clarified retentate fluid and resin mixture through the filtration module, thereby separating the mixture into a permeate fluid stream comprising the biologic product and a retentate stream comprising the resin; and recirculating the retentate fluid stream through the retentate fluid circuit for a second number of DVs while directing the permeate stream to a recovery container outside the fluid circuit, thereby isolating the biologic product in the recovery container.

Embodiment 1: A method for isolating a biologic product from a process fluid characterized by high cell density and/or high turbidity, comprising performing a capture operation by contacting the process fluid with a capture resin in a process vessel for a period of time sufficient to allow binding of the biologic product to the resin; performing a wash operation by circulating the process fluid and resin mixture through a filtration module, thereby separating the mixture into permeate and retentate fluid streams, wherein the filtration module and the process vessel are interconnected in a retentate fluid circuit, and recirculating the retentate fluid stream through the retentate fluid circuit for a first number of diafiltration volumes (DVs) while directing the permeate stream to a waste container outside the fluid circuit, thereby producing a clarified retentate fluid, and optionally concentrating the clarified retentate fluid in the process vessel; performing an elution operation by contacting the clarified retentate fluid with a volume of elution buffer for a period of time sufficient to allow disassociation of the biologic product from the resin; performing a harvest operation by circulating the clarified retentate fluid and resin mixture through the filtration module, thereby separating the mixture into a permeate fluid stream comprising the biologic product and a retentate fluid stream comprising the resin; and recirculating the retentate fluid stream through the retentate fluid circuit for a second number of DVs while directing the permeate stream to a recovery container outside the fluid circuit, thereby isolating the biologic product in the recovery container.

Embodiment 3: A method for isolating a biologic product from a process fluid characterized by high cell density and/or high turbidity, comprising performing a capture operation by contacting the process fluid with a capture resin in a process vessel for a period of time sufficient to allow binding of the biologic product to the resin while circulating the mixture of process fluid and resin through a filtration module, thereby separating the mixture into permeate and retentate fluid streams, wherein the filtration module and the process vessel are interconnected in a closed retentate fluid circuit and an open permeate fluid circuit such that the retentate fluid stream is prevented from exiting the filtration module, thereby retaining the resin, while the permeate fluid stream is directed through the permeate fluid circuit back to the process vessel; performing a wash operation by opening the retentate fluid circuit, circulating the process fluid and resin mixture through the filtration module and recirculating the retentate fluid stream through the retentate fluid circuit for a first number of diafiltration volumes (DVs) while closing the permeate fluid circuit and directing the permeate stream to a waste container outside the fluid circuit, thereby producing a clarified retentate fluid, and optionally concentrating the clarified retentate fluid in the process vessel; performing an elution operation by contacting the clarified retentate fluid with a volume of elution buffer for a period of time sufficient to allow disassociation of the biologic product from the resin; performing a harvest operation by circulating the clarified retentate fluid and resin mixture through the module, thereby separating the mixture into a permeate fluid stream comprising the biologic product and a retentate fluid stream comprising the resin; and recirculating the retentate fluid stream through the module for a second number of DVs while directing the permeate stream to a recovery container outside the fluid circuit, thereby isolating the biologic product.

Embodiment 4: A method for isolating a biologic product from a process fluid characterized by high cell density and/or high turbidity, comprising performing a capture operation by (i) contacting the process fluid with a capture resin in a primary process vessel for a period of time sufficient to allow binding of the biologic product to the resin; (ii) circulating the mixture of process fluid and resin for a second period of time through a secondary process vessel in fluid communication with a filtration module, thereby separating the mixture into permeate and retentate fluid streams, wherein the filtration module and the secondary process vessel are interconnected in a retentate fluid circuit and the primary process vessel, secondary process vessel, and filtration module are interconnected in a permeate fluid circuit, such that the retentate fluid stream is recirculated between the secondary process vessel and filtration module in the retentate fluid circuit while the permeate fluid stream is recirculated between the primary process vessel, the secondary process vessel, and the filtration module in the permeate fluid circuit; performing a wash operation by closing the permeate fluid circuit, circulating the mixture of process fluid and resin through the filtration module and recirculating the retentate fluid stream through the retentate fluid circuit for a first number of diafiltration volumes (DVs) while directing the permeate stream to a waste container outside the fluid circuit, thereby producing a clarified retentate fluid, and optionally concentrating the clarified retentate fluid in the secondary process vessel; performing an elution operation by contacting the clarified retentate fluid with a volume of elution buffer for a period of time sufficient to allow disassociation of the biologic product from the resin; and performing a harvest operation by circulating the clarified retentate fluid and resin mixture through the filtration module, thereby separating the mixture into a permeate fluid stream comprising the biologic product and a retentate fluid stream comprising the resin; and recirculating the retentate fluid stream through the retentate fluid circuit for a second number of DVs while directing the permeate stream to a recovery container outside the fluid circuit, thereby isolating the biologic product.

Embodiment 5: The method of Embodiment 2, wherein the method comprises performing the capture operation in a secondary vessel fluidly connected to the process vessel, the method comprising transferring a first volume of process fluid from the process vessel into the secondary vessel, wherein the secondary vessel contains the resin, or the resin is added to the secondary vessel, and performing the capture operation and wash operation to obtain a clarified retentate fluid in the secondary vessel.

Embodiment 6: The method of Embodiment 5, wherein the method comprises performing a second or further capture operation prior to the elution operation by transferring a second or further volume of process fluid into the secondary process vessel containing the clarified retentate fluid and performing a second or further capture operation and wash operation to obtain a second or further clarified retentate fluid in the secondary vessel, optionally repeating the capture and wash operations with a third or further volume of process fluid prior to performing the elution and harvest operations.

Embodiment 7: The method of any one of Embodiments 1 to 6, wherein during one or both of the wash and harvest operations, fluid lost to the permeate stream is replaced to maintain a constant volume of fluid in the process vessel and/or the secondary vessel in either a batch or continuous process.

Embodiment 8: The method of any one of claims 1 to 7, wherein during one or both of the wash and harvest operations, fluid lost to the permeate stream is not replaced in order to concentrate the fluid in the process vessel.

Embodiment 9: The method of any one of Embodiments 1 to 8, wherein the process fluid is characterized by a viable cell density (VCD) or total cell density (TCD) of from 10E5 to 10E9 cells/ml for insect or mammalian cells, or by an optical density (OD) of from 1-350 at 600 or 620 nanometers (nm) for bacterial cells.

Embodiment 10: The method of any one of Embodiments 1 to 9, wherein the process fluid is characterized by a turbidity of from 100-30,000 nephelometric turbidity units (NTUs) or from 200-1,000 NTU before contacting with the resin.

Embodiment 11: The method of any one of Embodiments 1 to 10, wherein the process fluid is characterized by a viscosity of from about 1.5-30 centipoise (cP).

Embodiment 12: The method of any one of Embodiments 1 to 11, wherein the wash operation is sufficient to remove 95-99% of cells and/or cellular proteins and nucleic acids from the retentate fluid stream.

Embodiment 13: The method of any one of Embodiments 1 to 11, wherein the wash operation is sufficient to achieve a 2-5 average log reduction of cellular proteins and nucleic acids in the retentate fluid stream.

Embodiment 14: The method of any one of Embodiments 1 to 13, wherein following the harvest operation, at least 90% of the resin is retained in the retentate fluid.

Embodiment 15: The method of any one of Embodiments 1 to 14, wherein following the harvest operation, the harvested biologic product is subjected to filtration through a tangential flow depth filtration (TFDF) filter medium.

Embodiment 16: The method of any one of Embodiments 1 to 15, wherein the filtration module comprises a tangential flow depth filtration (TFDF) filter medium comprising one or a plurality of hollow fiber elements forming a hollow fiber depth filter medium, each hollow fiber element consisting of a porous wall having a thickness of from 2-10 millimeters (mm) defining a lumen having an internal diameter (ID) of from 1-12 mm, a porosity of from about 50-90%, and a pore rating of from 10-50 microns.

Embodiment 17: The method of Embodiment 16, wherein each of the one or a plurality of hollow fiber elements forming the hollow fiber depth filter medium consists of a porous wall having a thickness of from 2-10 mm defining a lumen having an ID of from 1-12 mm, a porosity of from about 60-90%, and a pore rating of 10, 20, 30, 40, or 50 microns.

Embodiment 18: The method of any one of Embodiments 1 to 15, wherein the filtration module comprises a large porosity tangential flow filtration (TFF) filter medium constructed of nonwoven fibers having pore size in the range of 50-200 microns.

Embodiment 19: The method of Embodiment 18, wherein the filter medium is constructed from a nonwoven polypropylene/polyethylene polymer, a polyester polymer, a polyamide polymer, or a fluoropolymer.

Embodiment 20: The method of Embodiment 19, wherein the filter medium includes a porous wall having a thickness of from 0.1 to 0.5 mm defining a lumen having an ID of from 1-12 mm and a porosity of from about 60-90%.

Embodiment 21: The method of Embodiment 20, wherein the filter medium in the form of a flat sheet spiral wound into a tubular form, optionally thermally or ultrasonically welded to itself in a spiral pattern to form a tubular filter element.

Embodiment 22: The method of Embodiment 21, wherein the filter medium is manufactured using wetlaid technology.

Embodiment 23: The method of Embodiment 22, wherein the filter medium is not formed by extrusion.

Embodiment 24: The method of any one of Embodiments 1 to 19, wherein the biologic product is an antibody, a recombinant protein or a virus particle.

Embodiment 25: The method of Embodiment 24 wherein the resin is functionalized with Fc-binding ligands or ligands that bind to virus particles.

Embodiment 26: A system for isolating a biologic product from a process fluid characterized by high cell density and/or high turbidity, comprising a process vessel containing the process fluid; at least one filtration module in fluid communication with the process vessel, the filtration module comprising one or a plurality of hollow fiber elements forming a filter medium, each hollow fiber element consisting of a porous wall having a thickness of from 0.5-10 millimeters (mm) defining a lumen having an internal diameter (ID) of from 1-12 mm, a porosity of from about 50-90%, and a pore rating of from 10-50 microns, and a housing adapted to separate the process fluid into permeate and retentate fluid streams as it flows through the filter medium, the housing provided with an inlet, a permeate outlet and a retentate outlet; and at least one pump.

Embodiment 27: The system of Embodiment 26, wherein at least two filtration modules are connected in series or in parallel with the process vessel, optionally wherein two or more units comprising a filtration module and process vessel are connected in series or in parallel.

Embodiment 28: The system of Embodiment 26, wherein the system comprises a retentate pump and a permeate pump.

Embodiment 29: The system of Embodiment 26, wherein the system comprises a permeate container and a product recovery container.

Embodiment 30: The system of Embodiment 26, wherein the system comprises a supply of fluid or buffer joined in a feed relationship to the process vessel.

Embodiment 31: A tangential flow filtration (TFF) or tangential flow depth filtration (TFDF) module comprising one or a plurality of hollow fiber elements forming a hollow fiber filter medium or hollow fiber depth filter medium, each hollow fiber element consisting of a porous wall defining a lumen having an internal diameter (ID) of from 1-12 mm, a porosity of from about 50-90%, and a pore rating of from 10-50 microns, and a housing adapted to separate a process fluid into permeate and retentate fluid streams as it flows through the filter medium, the housing provided with an inlet, a permeate outlet and a retentate outlet.

32: A tangential flow filtration (TFF) module comprising a filter medium of a nonwoven polypropylene/polyethylene polymer in the form of a flat sheet spiral wound into a tubular form and having a pore size of from 50-200 microns, a porous wall having a thickness of from 0.1 to 0.5 mm defining a lumen having an internal diameter (ID) of from 1-12 millimeters, a porosity of from about 60-90%, and a pore rating of 40, 50, 100, 150, or 200 microns, and a housing adapted to separate a process fluid into permeate and retentate fluid streams as it flows through the filter medium, the housing provided with an inlet, a permeate outlet and a retentate outlet.

Embodiment 33: A system for isolating a biologic product from a process fluid characterized by high cell density and/or high turbidity, comprising a process vessel containing the process fluid, at least one tangential flow filtration (TFF) module in fluid communication with the process vessel, the filtration module comprising a filter medium of a nonwoven polypropylene/polyethylene polymer in the form of a flat sheet spiral wound into a tubular form and having a pore size of from 50-200 microns, a porous wall having a thickness of from 0.1 to 0.5 mm defining a lumen having an internal diameter (ID) of from 1-12 millimeters, a porosity of from about 60-90%, and a pore rating of 40, 50, 100, 150, or 200 microns, and a housing adapted to separate a process fluid into permeate and retentate fluid streams as it flows through the filter medium, the housing provided with an inlet, a permeate outlet and a retentate outlet, a recirculation loop comprising flexible tubing interconnected between the process vessel and the TFF module to form a retentate fluid circuit, and at least one pump.

Embodiment 34: The system of Embodiment 33, wherein at least two TFF modules are connected in series or in parallel with the process vessel, optionally wherein two or more units comprising a filtration module and process vessel are connected in series or in parallel.

Embodiment 35: The system of Embodiment 33 or 34, wherein the system comprises a feed pump, a retentate pump and a permeate pump.

Embodiment 36: The system of any one of Embodiments 33 to 35, wherein the system comprises one or more of a wash buffer, elution buffer, and regeneration buffer joined in a feed relationship to the recirculation loop.

While the invention herein disclosed has been described by means of specific embodiments and applications thereof, numerous modifications and variations could be made thereto by those skilled in the art without departing from the scope of the invention set forth in the claims.

It will be appreciated that the present invention is set forth in various levels of detail in this application. In certain instances, details that are not necessary for one of ordinary skill in the art to understand the invention, or that render other details difficult to perceive may have been omitted. The terminology used herein is for the purpose of describing particular embodiments only and is not intended to be limiting beyond the scope of the appended claims. Unless defined otherwise, technical terms used herein are to be understood as commonly understood by one of ordinary skill in the art to which the disclosure belongs.

Various features of a process system may be used independently of, or in combination, with each other. It will be appreciated that a system as disclosed herein may be embodied in different forms and should not be construed as limited to the illustrated embodiments of the figures.

It should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with one or more other features to yield a still further embodiment. Thus, it is intended that the present subject matter covers such modifications and variations as come within the scope of the appended claims and their equivalents.

In view of the above, it should be understood that the various embodiments illustrated in the figures have several separate and independent features, which each, at least alone, has unique benefits which are desirable for, yet not critical to, the presently disclosed vessel, system, and associated method. Therefore, the various separate features described herein need not all be present in order to achieve at least some of the desired characteristics and/or benefits described herein.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The term “about” refers to a range of 1-10% around the stated value. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another.

In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A method for isolating a biologic product from a process fluid characterized by high cell density and/or high turbidity, comprising
performing a capture operation by contacting the process fluid with a capture resin in a retentate fluid circuit of a filtration module for a period of time sufficient to allow binding of the biologic product to the resin;
performing a wash operation by separating the process fluid and resin mixture into permeate and retentate fluid streams of the filtration module and recirculating the retentate fluid stream through the retentate fluid circuit for a first number of diafiltration volumes (DVs) while directing the permeate stream to a waste container outside the fluid circuit, thereby producing a clarified retentate fluid, and optionally concentrating the clarified retentate fluid in the retentate fluid circuit;
performing an elution operation by contacting the clarified retentate fluid with a volume of elution buffer in the retentate fluid circuit for a period of time sufficient to allow disassociation of the biologic product from the resin;
performing a harvest operation by circulating the clarified retentate fluid and resin mixture through the filtration module, thereby separating the mixture into a permeate fluid stream comprising the biologic product and a retentate stream comprising the resin; and
recirculating the retentate fluid stream through the retentate fluid circuit for a second number of DVs while directing the permeate stream to a recovery container outside the fluid circuit, thereby isolating the biologic product in the recovery container.

2. The method of claim 1, wherein during one or both of the wash and harvest operations, fluid lost to the permeate stream is replaced to maintain a constant volume of fluid in the process vessel and/or the secondary vessel in either a batch or continuous process.

3. The method of claim 1, wherein during one or both of the wash and harvest operations, fluid lost to the permeate stream is not replaced in order to concentrate the fluid in the process vessel.

4. The method of claim 1, wherein the process fluid is characterized by a viable cell density (VCD) or total cell density (TCD) of from 10E5 to 10E9 cells/ml for insect or mammalian cells, or by an optical density (OD) of from 1-350 at 600 or 620 nanometers (nm) for bacterial cells.

5. The method of claim 1, wherein the process fluid is characterized by a turbidity of from 100-30,000 nephelometric turbidity units (NTUs) or from 200-1,000 NTU before contacting with the resin.

6. The method of claim 1, wherein the process fluid is characterized by a viscosity of from about 1.5-30 centipoise (cP).

7. The method of claim 1, wherein the wash operation is sufficient to remove 95-99% of cells and/or cellular proteins and nucleic acids from the retentate fluid stream.

8. The method of claim 1, wherein the wash operation is sufficient to achieve a 2-5 average log reduction of cellular proteins and nucleic acids in the retentate fluid stream.

9. The method of claim 1, wherein following the harvest operation, at least 90% of the resin is retained in the retentate fluid.

10. The method of claim 1, wherein following the harvest operation, the harvested biologic product is subjected to filtration through a tangential flow depth filtration (TFDF) filter medium.

11. The method of claim 1, wherein the filtration module comprises a tangential flow depth filtration (TFDF) filter medium comprising one or a plurality of hollow fiber elements forming a hollow fiber depth filter medium, each hollow fiber element consisting of a porous wall having a thickness of from 2-10 millimeters (mm) defining a lumen having an internal diameter (ID) of from 1-12 mm, a porosity of from about 50-90%, and a pore rating of from 10-50 microns.

12. The method of claim 11, wherein each of the one or a plurality of hollow fiber elements forming the hollow fiber depth filter medium consists of a porous wall having a thickness of from 2-10 mm defining a lumen having an ID of from 1-12 mm, a porosity of from about 60-90%, and a pore rating of 10, 20, 30, 40, or 50 microns.

13. The method of claim 1, wherein the filtration module comprises a large porosity tangential flow filtration (TFF) filter medium constructed of nonwoven fibers having pore size in the range of 50-200 microns.

14. The method of claim 13, wherein the filter medium is constructed from a nonwoven polypropylene/polyethylene polymer, a polyester polymer, a polyamide polymer, or a fluoropolymer.

15. The method of claim 14, wherein the filter medium includes a porous wall having a thickness of from 0.1 to 0.5 mm defining a lumen having an ID of from 1-12 mm and a porosity of from about 60-90%.

16. The method of claim 15, wherein the filter medium in the form of a flat sheet spiral wound into a tubular form, optionally thermally or ultrasonically welded to itself in a spiral pattern to form a tubular filter element.

17. The method of claim 16, wherein the filter medium is manufactured using wetlaid technology.

18. The method of claim 17, wherein the filter medium is not formed by extrusion.

19. The method of claim 1, wherein the biologic product is an antibody, a recombinant protein or a virus particle.

20. The method of claim 19 wherein the resin is functionalized with Fc-binding ligands or ligands that bind to virus particles.

21. A tangential flow filtration (TFF) module comprising
a filter medium of a nonwoven polypropylene/polyethylene polymer in the form of a flat sheet spiral wound into a tubular form and having a pore size of from 50-200 microns, a porous wall having a thickness of from 0.1 to 0.5 mm defining a lumen having an internal diameter (ID) of from 1-12 millimeters, a porosity of from about 60-90%, and a pore rating of 40, 50, 100, 150, or 200 microns, and
a housing adapted to separate a process fluid into permeate and retentate fluid streams as it flows through the filter medium, the housing provided with an inlet, a permeate outlet and a retentate outlet.

22. A system for isolating a biologic product from a process fluid characterized by high cell density and/or high turbidity, comprising
a process vessel containing the process fluid,
at least one tangential flow filtration (TFF) module in fluid communication with the process vessel, the filtration module comprising
a filter medium of a nonwoven polypropylene/polyethylene polymer in the form of a flat sheet spiral wound into a tubular form and having a pore size of from 50-200 microns, a porous wall having a thickness of from 0.1 to 0.5 mm defining a lumen having an internal diameter (ID) of from 1-12 millimeters, a porosity of from about 60-90%, and a pore rating of 40, 50, 100, 150, or 200 microns, and a housing adapted to separate a process fluid into permeate and retentate fluid streams as it flows through the filter medium, the housing provided with an inlet, a permeate outlet and a retentate outlet,
a recirculation loop comprising flexible tubing interconnected between the process vessel and the TFF module to form a retentate fluid circuit, and
at least one pump.

23. The system of claim 22, wherein at least two TFF modules are connected in series or in parallel with the process vessel, optionally wherein two or more units comprising a filtration module and process vessel are connected in series or in parallel.

24. The system of claim 22, wherein the system comprises a feed pump, a retentate pump and a permeate pump.

25. The system of claim 22, wherein the system comprises one or more of a wash buffer, elution buffer, and regeneration buffer joined in a feed relationship to the recirculation loop.
